(19) United States
(12) Patent Application Publication
(10) Pub. No.: US 2013/0228177 A1
(43) Pub. Date: Sep. 5, 2013

(54) CPAP HUMIDIFIER TANK CONTROL VALVE

(71) Applicant: Gerald Schueller, Bloomington, MN (US)

(72) Inventor: Gerald Schueller, Bloomington, MN (US)

(21) Appl. No.: 13/409,588

(22) Filed: Mar. 1, 2012

(51) Int. Cl. A61M 16/06; A61M 16/00; A61M 16/16

(52) U.S. Cl. 128/203.19; 261/65; 261/30; 261/28; 261/27; 128/203.29

(57) ABSTRACT

A CPAP humidifier system includes an air pump, a humidifier tank, a control valve, and an auxiliary reservoir. The humidifier tank has an air inlet opening and an air outlet opening. The air inlet opening fluidly connects to the air pump, and the air outlet opening fluidly connects to an air mask. The control valve is attached to the air outlet opening and defines both an air outlet passageway and a liquid inlet passageway. The auxiliary reservoir fluidly connects to the liquid inlet passageway for providing liquid to the humidifier tank.

### BACKGROUND

The present disclosure relates generally to Continuous Positive Airway Pressure (CPAP) machines, and more particularly to refilling humidifier tanks for CPAP machines.

Sleep apnea is a sleeping disorder characterized by pauses in breathing during sleep. Patients with sleep apnea often benefit from nighttime respiratory assistance with a CPAP machine. CPAP machines are most commonly used for home treatment of sleep apnea, but also can be used for hospital patients who are ill or for newborn infants. CPAP machines work by continuously flowing pressurized air through a patient's throat to keep the airway open. The CPAP air is usually drawn from the surrounding atmosphere and consequently, can be quite dry. Some CPAP machines include a humidifier for adding moisture to the pressurized air, but conventional CPAP humidifiers are less than ideal.

### SUMMARY

A respiratory humidifier includes a tank, an air inlet, an air outlet, and a control valve. The tank stores liquid. The air inlet opening is located on the tank and fluidly connects the tank to an air pump. The air outlet opening is located on the tank and fluidly connects the tank to an air mask. The control valve is attached to the air outlet opening and defines both an air outlet passageway and a liquid inlet passageway. The control valve includes a shut-off mechanism for blocking the liquid inlet passageway when liquid in the tank reaches a level.

A CPAP humidifier system includes an air pump, a humidifier tank, a control valve, and an auxiliary reservoir. The humidifier tank has an air inlet opening and an air outlet opening. The air inlet opening is fluidly connected to the air pump, and the air outlet opening is fluidly connected to an air mask. The control valve is attached to the air outlet opening and defines both an air outlet passageway and a liquid inlet passageway. The auxiliary reservoir is fluidly connected to the liquid inlet passageway for providing liquid to the humidifier tank.

A method of providing humidified air to a user includes pumping air and flowing pumped air to a humidifier tank to form humidified air. The method further includes flowing humidified air from the humidifier tank through a control valve to a mask and flowing liquid from an auxiliary tank through the control valve to the humidifier tank.

### BRIEF DESCRIPTION OF THE DRAWINGS

FIG. 1 is a schematic view of a CPAP humidifier system in accordance with the present disclosure.

FIG. 2 is perspective view of a humidifier tank having a control valve.

FIG. 3 is a vertical cross sectional view of the humidifier tank and control valve of FIG. 2.

FIG. 4A is a vertical cross sectional view, and FIG. 4B is a horizontal cross sectional view, of a first embodiment of a control valve.

FIG. 5A is a vertical cross sectional view, and FIG. 5B is a horizontal cross sectional view, of a second embodiment of a control valve.

FIG. 6A is a vertical cross sectional view, and FIG. 6B is a horizontal cross sectional view, of a third embodiment of a control valve.

FIG. 7A is a vertical cross sectional view, FIG. 7B is a horizontal cross sectional view, and FIG. 7C is a vertical cross sectional view of a fourth embodiment of a control valve.

FIG. 8A is a vertical cross sectional view, FIG. 8B is a horizontal cross sectional view, and FIG. 8C is an exploded cross sectional view of a fifth embodiment of a control valve.

FIG. 9 is an exploded cross sectional view of a sixth embodiment of a control valve.

FIG. 10A is an exploded cross sectional view, and FIG. 10B is an assembled cross sectional view, of a seventh embodiment of a control valve.

FIG. 11A is an exploded cross sectional view, and FIG. 11B is an assembled cross sectional view, of an eight embodiment of a control valve.

FIG. 12 is a vertical cross sectional view of a humidifier tank having a drop in control valve.

FIG. 13A is a side view, and FIG. 13B is a front view, of a flexible auxiliary liquid reservoir.

FIG. 14A is a side view of a first embodiment of an auxiliary liquid reservoir and electric pump.

FIG. 14B is a side view of a second embodiment of an auxiliary liquid reservoir and electric pump.

FIG. 14C is a side view of a third embodiment of an auxiliary liquid reservoir and electric pump.

FIG. 14D is a side view of a fourth embodiment of an auxiliary liquid reservoir and electric pump.

### DETAILED DESCRIPTION

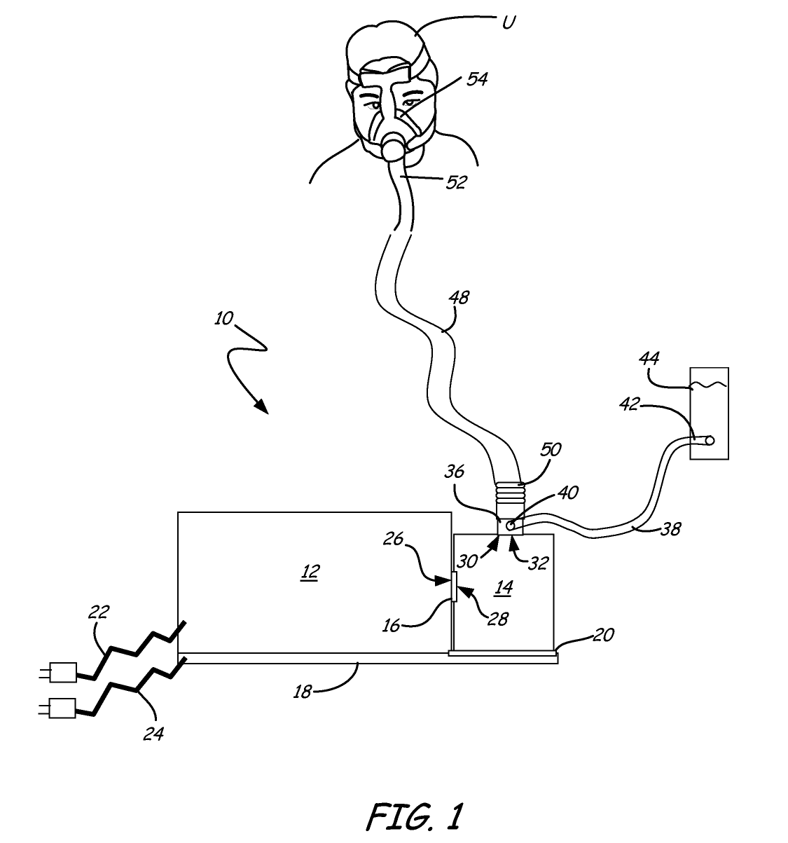

Some CPAP machines incorporate humidifiers for adding moisture to CPAP air. Conventional CPAP humidifier tanks are designed to hold only enough water for about eight to ten hours of CPAP use (e.g. a few ounces of water) and therefore, requires liquid refilling on a daily basis. The present disclosure proposes use of an auxiliary liquid reservoir connected to a CPAP humidifier tank by a control valve. The auxiliary reservoir holds anywhere from three days to three weeks worth of liquid and therefore, negates the need for daily refills of the CPAP humidifier tank. The control valve regulates flow of liquid from the auxiliary reservoir to the CPAP humidifier tank based on a level of liquid within the humidifier tank. The proposed CPAP humidifier system is described in detail below with reference to FIGS. 1-14D.

FIG. 1 is a schematic view of CPAP humidifier system 10 in accordance with the present disclosure. CPAP humidifier system 10 includes air pump 12, water tank 14, air conduit 16, base 18, heater 20, first plug 22, second plug 24, pump air outlet 26, tank air inlet 28, tank air outlet 30, tank liquid inlet 32, control valve 36 liquid conduit 38 having first end 40 and second end 42, auxiliary reservoir 44, humidified air conduit 48 having first end 50 and second end 52, and mask 54. Air pump 12 pumps air through humidifier tank 14 for increasing humidity of the air before it is delivered to user U. Humidifier tank 14 is connected to auxiliary reservoir 44 through control valve 36, which regulates an amount of liquid within humidifier tank 14.

In the depicted CPAP humidifier system 10, air pump 12 is positioned next to humidifier tank 14. Air conduit 16 connects a side of air pump 12 to an adjacent side of humidifier tank 14. Both air pump 12 and humidifier tank 14 rest on top of base 18, which is usually in contact with a floor or other surface in a room. Heater 20 is located between a bottom of humidifier tank 14 and a top of base 18. First plug 22 electrically connects air pump 12 to a power source (e.g. an outlet) and second plug 24 electrically connects heater 20 (through base 18) to a power source (e.g. an outlet). Air pump 12 includes pump air outlet 26 and humidifier tank 14 inlets tank air inlet 28, which are fluidly connected to one another by air conduit 16. Humidifier tank 14 further includes tank air outlet 30 and tank liquid inlet 32, which are commonly located at a top of humidifier tank 14.

When activated or “turned on”, air pump 12 uses electrical energy supplied by first plug 22 to pressurize ambient air. The pressurized air is pumped from air pump 12 to humidifier tank 14. More specifically, pressurized air exits pump air outlet 26 and travels through air conduit 16 to tank air inlet 28. When activated or “turned on”, heater 20 uses electrical energy supplied by second plug 24 to vaporize liquid (e.g. turn water into steam). The pressurized air mixes with the vapor to form humidified air within humidifier tank 14. This humidified air then exits humidifier tank 14 at tank air outlet 30 for use by user U.

Control valve 36 is attached to commonly located tank air outlet 30 and tank liquid inlet 32. Liquid conduit 38 fluidly connects auxiliary reservoir 44 to humidifier tank 14. More specifically, liquid conduit 38 includes first end 40 attached to control valve 36 and second end 42 attached to auxiliary reservoir 44. A top of control valve 36 is attached to first end 50 of humidified air conduit 48, and a bottom of control valve 36 is attached to tank air outlet 30 and tank liquid inlet 32. Humidified air conduit 48 fluidly connects humidifier tank 14 to mask 54. More specifically, humidified air conduit 48 includes first end 50 attached to control valve 36 and second end 52 attached to mask 54. Mask 54 is configured to be secured to a face of user U.

Liquid is stored within auxiliary reservoir 44 for supplying liquid to and refilling humidifier tank 14. Auxiliary reservoir 44 can take many different forms (e.g. flexible or rigid, associated with an electric pump or using gravity, etc.). Fluid flows out of auxiliary reservoir 44, through liquid conduit 38, and through control valve 36 to liquid inlet 32 of tank humidifier 14. For example, auxiliary reservoir can hold between about 0.125-1 gallon (4.73 milliliters-3.8 liters) of fluid, while humidifier tank 14 can hold between about 2-10 ounces (54.1-295.7 milliliters) of fluid. Accordingly, auxiliary reservoir 44 of CPAP humidifier system 10 requires refilling far less frequently than conventional CPAP systems possessing only a tank humidifier and lacking an auxiliary reservoir 44. Humidified air flows out of tank air outlet 30, through control valve 36, through adaptor 46, and through humidified air conduit 48 to mask 54 for inhalation by user U. With CPAP humidifier system 10, it is possible to flow both liquid and humidified air through control valve 36 simultaneously. Control valve 36 defines both an air passageway and a liquid passageway as described in more detail below.

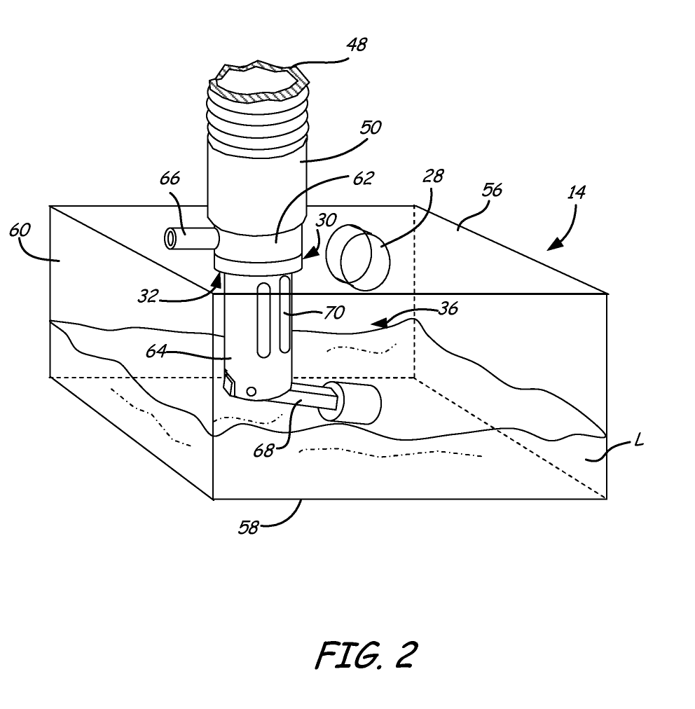

FIG. 2 is perspective view of humidifier tank 14 having control valve 36 attached to first end 50 of humidified air conduit 48. Humidifier tank 14 includes tank air inlet 28, tank air outlet 30, tank liquid inlet 32, top 56, bottom 58, four walls 60, and liquid L. Control valve 36 includes top portion 62, bottom portion 64, liquid inlet port 66, shut-off mechanism 68, and air vents 70. Control valve 36 is attached to humidifier tank 14 to mange liquid intake from an auxiliary reservoir (e.g auxiliary reservoir 44 in FIG. 1), while simultaneously allowing a flow of humidified air to first end 50 of humidified air conduit 48.

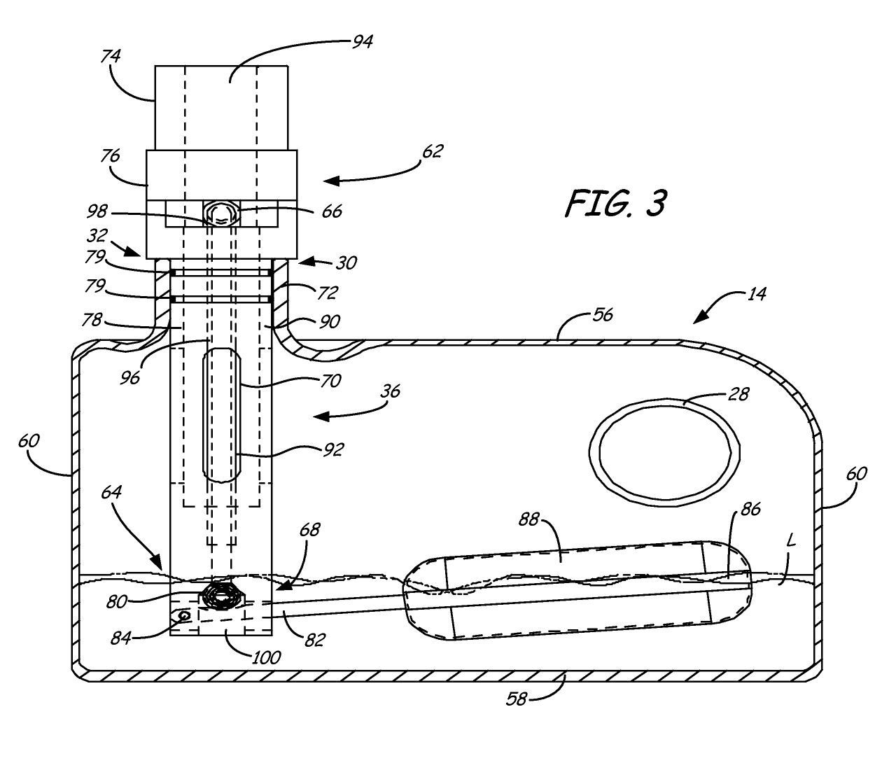

In the depicted embodiment, humidifier tank 14 is transparent to show its interaction with control valve 36. Humidifier tank 14 is a substantially rectangular box and includes top 56, opposite bottom 58, and four side walls 60. Tank air inlet 28 is an opening located on one side wall 60 of humidifier tank, and is fluidly connected to an air pump (e.g. air pump 12 in FIG. 1). In the depicted embodiment, tank air outlet 30 and tank liquid inlet 32 are a single opening located on top 56 of humidifier tank 14, which is surrounded by a raised collar (e.g. see collar 72 in FIG. 3). For alternative embodiments, tank air outlet 30 and tank liquid inlet 32 are separate openings and one or both of these openings can be located on side wall 60 of humidifier tank 14. In a conventional CPAP system, a tank air outlet is directly connected to a humidified air conduit, and there is no tank liquid inlet or control valve. In the presently described embodiment, control valve 36 is attached to tank air outlet 30 to repurpose this space into both tank air outlet 30 and tank liquid inlet 32.

Control valve 36 includes top portion 62, and an opposite bottom portion 64. Top portion 62 has first end attached to first end 50 of humidified air conduit 48 and a second end extending through tank air outlet 30 and tank liquid inlet 32. Bottom portion 64 extends from its attachment to top portion 62 near tank air outlet 30 and tank liquid inlet 32 down into humidifier tank 14. Liquid inlet port 66 projects from a side of top portion 62 for connection to a liquid conduit (e.g. first end 40 of liquid conduit 38 from FIG. 1). Shut-off mechanism extends 68 from a bottom of bottom portion 64 and is in contact with liquid L within humidifier tank 14. Vents 70 are oblong openings extending vertically along bottom portion 62.

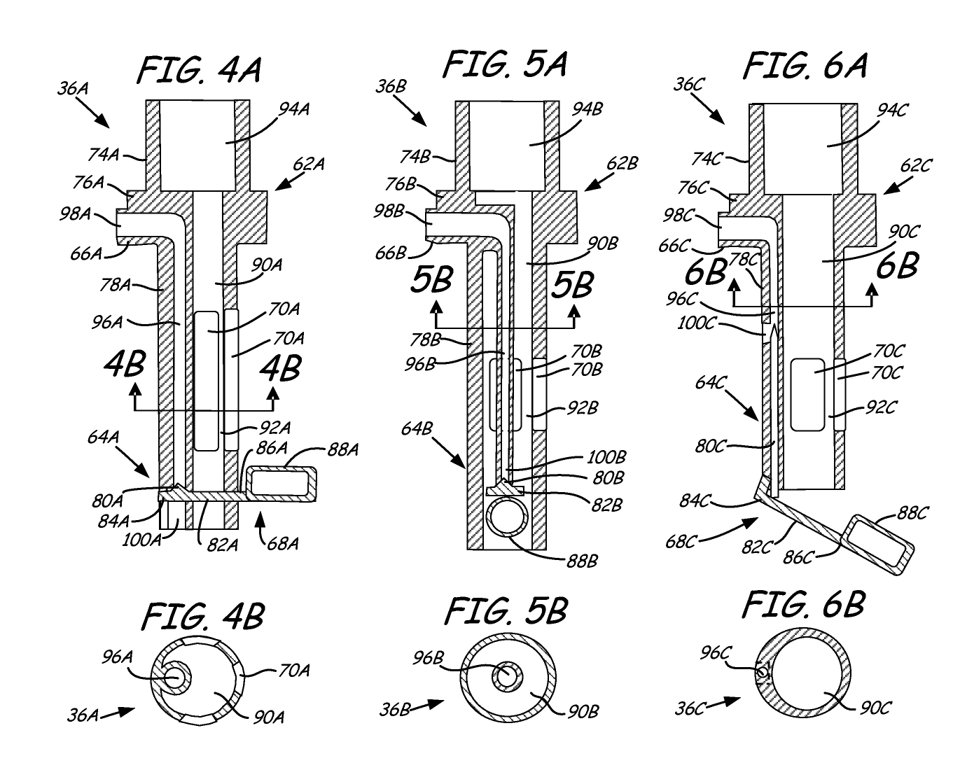

Pressurized air flows into humidifier tank 14 from tank air inlet 28. Liquid flows from liquid port 66, through top portion 62 (extending through tank liquid inlet 32) and bottom portion 64 downwardly into humidifier tank 14. Liquid L collects on bottom 58 of humidifier tank 14 and is contained by side walls 60. Pressurized air within humidifier tank 14 mixes with vapor to form humidified air. The humidified air flows into vents 70 in bottom portion 62, upwardly through upper portion 60 (extending through tank air outlet 30) and to first end 50 of humidified air conduit 48. The design of control valve 36 allows the outward flow of humidified air can to occur simultaneously with the inward flow of liquid. When liquid L within humidifier tank 14 reaches a set level, shut-off mechanism 68 is activated to slow or halt the inward flow of liquid L through control valve 36. In the depicted embodiment, shut-off mechanism 68 includes a float arm and a seal. Alternative designs for control valve 36 including alternative shut-off mechanisms 68 are contemplated and discussed in detail below with reference to FIGS. 4A-12.

FIG. 3 is a vertical cross sectional view of humidifier tank 14 having a control valve 36 with humidified air conduit 48 removed. Humidifier tank 14 includes tank air inlet 28, tank air outlet 30, tank liquid inlet 32, top 56, bottom 58, walls 60, and liquid L. Control valve 36 includes top portion 62, bottom portion 64, liquid inlet port 66, shut-off mechanism 68, and air vents 70. Humidifier tank 14 further includes collar 72. Control valve 36 further includes neck 74, shoulder 76, body 78, O-rings 79, seal 80, arm 82 having first end 84 and second end 86, and float 88. Control valve 36 defines air passageway 90 having inlet 92 and outlet 94, and liquid passageway 96 having inlet 98 and outlet 100. Control valve 36 simultaneously allows a flow of humidified air to through air passageway 90 and a flow of liquid through liquid passageway 90.

Substantially horizontal top 56 of humidifier tank 14 includes a vertically raised collar 72, which defines a common, circular, opening for both tank air outlet 30 and tank liquid inlet 32. A central portion of control valve 36 extends through collar 72 such that top portion 62 is located vertically above collar 72 and outside of humidifier tank 14 while bottom portion 64 is located vertically below collar 72 and inside of humidifier tank 14. Upper portion 62 of control valve 36 can be further divided into neck 74 and shoulder 76. Shoulder 76 and neck 74 can be manufactured as individual items and later joined to from upper portion 62, or they can be manufactured as a single piece. Neck 74 is the topmost portion of upper portion 62 and is sized to mate with (e.g. fit inside of) a humidifier conduit (such as first end 50 of humidifier conduit 48 shown in FIG. 2). Shoulder 76 has a top end attached to neck 74, and a bottom end abutting a top of collar 72. Shoulder 76 has a diameter larger than neck 74 and collar 72 such that a bottom end of shoulder 76 rests on top of collar 72 and prevents control valve 36 from falling into humidifier tank 14. Liquid inlet port 66 extends into a side of shoulder 76.

Neck 74, shoulder 76, and body 78 are substantially cylindrical, coaxial and vertically stacked in series. In alternative embodiments, neck 74, shoulder 76, and body 78 are rectangular and offset from each other. As shown in FIG. 3, shoulder 76 has a largest relative diameter, neck 74 has a middle sized relative diameter, and body 78 has a smallest relative diameter. Body 78 extends vertically from upper portion 62 to lower portion 64 of control valve 36. A top end of body 78 is attached to a bottom end of shoulder 76 and forms a lip. Body 78 has a diameter smaller than both shoulder 76 and collar 72, such that body 78 extends through collar 72 into humidifier tank 14. An outside surface of body 78 and an inside surface of collar 72 can form a light interference fit. Body 78 is the “fitting portion” of control valve 36. One or more o-rings 79 can be located at a top end of body 78 for providing frictional contact between an outer surface of body 78 and an inside surface of collar 72. Located more toward a center of body 78 are a plurality of oblong air vents 70 for receiving air into control valve 36. A bottom end of body 78 is located at lower end 64 of control valve 36 is attached to shut-off mechanism 68.

As shown in FIG. 3, each portion of shut-off mechanism 68 is in contact with liquid L. In the depicted embodiment, shut-off mechanism 68 be further divided into seal 80, arm 82, and float 88. Seal 80 is a spherical ball located inside of an opening in the lower end of body 78. Arm 82 extends horizontally from a lower end of body 78 at a location near seal 80. More specifically, arm 82 extends from a first end 84 secured to a lower end of body 78 to second end 86 spaced a horizontal distance away from seal 80. Seal 80 can be made of a resilient material such as rubber or the like. In the depicted embodiment, arm 82 is substantially rectangular and second end 86 is surrounded by substantially cylindrical float 88. As indicated by its name, float 88 is configured to float such that a top of float 88 is located above a surface of liquid L. Float 88 can be made of a light weight material having a density less than water. In alternative embodiments, float 88 is a hollow structure.

Control valve 36 defines substantially cylindrical air passageway 90. Air passageway 90 is defined by wall that extends through body 78, shoulder 76, and neck 74 of control valve 36. Inlet 92 of air passageway 90 is located centrally on body 78 near air vents 70. Outlet 94 of air passageway 90 is located at a top end of neck 74. Humidified air from humidifier tank 14 flows into inlet 92 of air passageway 90 through air vents 70. Humidified air flows upwardly from inlet 92 along air passageway 90 to outlet 94 in order to exit humidifier tank 14 (air leaves tank air outlet 30 through control valve 36). As described with reference to FIG. 1, humidified air conduit 48 is attached to control valve 36 for conducting humidified air from humidifier tank 14 to mask 54 for use by user U. More specifically, first end 50 of humidified air conduit 48 is attached to neck 74, such that outlet 94 of air passageway 90 is fluidly connected to mask 54. Air passageway 90 is continuously open during both inspiration and exhalation of user U.

Control valve 36 also defines substantially cylindrical liquid passageway 96. Liquid passageway 96 is defined by wall that extends through shoulder 76 and body 78 to shut-off mechanism 68 of control valve 36. Inlet 98 of liquid passageway is located centrally within shoulder 76 near liquid inlet port 66. Outlet 100 of liquid passageway 96 is located at lower portion 64 or bottom end of body 78 near shut-off mechanism 68. Liquid flows from a conduit attached to a liquid reservoir (such as liquid conduit 38 and liquid reservoir 44 from FIG. 1) into inlet 98 of liquid passageway 96 through liquid inlet port 66. Liquid flows downwardly from inlet 98 along liquid passageway 96 to outlet 100 in order to enter humidifier tank 14 (liquid enters tank liquid inlet 32 through control valve 36). In the depicted embodiment, liquid passageway 96 is located concentrically within air passageway 90 although other configurations are contemplated (e.g. see FIGS. 4A-12).

Shut-off mechanism 68 is located at outlet 100 of liquid passageway 96. In the depicted embodiment, outlet 100 of liquid passageway 96 is slightly expanded in relation to the rest of liquid passageway 96 and seal 80 is located in this expanded portion of outlet 100. As shown in FIG. 3, when liquid L levels are relatively high in humidifier tank 14, float 88 causes arm 82 to raise and form an acute angle with liquid passageway 96. When arm 82 is raised it pushes seal 80 upwardly into, and forms a seal with, outlet 100 of liquid passageway 96. Liquid will flow into inlet 98 and fill passageway 96, but not exit outlet 100 due to seal formed by arm 82 and seal 80. In other words, shut-off mechanism 68 shuts off the flow of liquid into humidifier tank 14 when liquid level L reaches a set level. The contrast is also true.

As liquid L is depleted from vaporization, float 88 will move downwardly with a surface of liquid L. The lowered float will cause arm 82 to drop and form a perpendicular angle or an obtuse angle with liquid passageway 96. Once arm 82 drops, it no longer forces seal 80 against outlet 100 and seal 80 can drop down out of its sealing arrangement with liquid passageway 96. Liquid will be free to flow out of outlet 100 and into humidifier tank 14 to replenish the depleted liquid L level. Accordingly, shut-off mechanism 68 responds to a level of liquid L in tank 14 by either blocking or allowing incoming liquid flow. The control over liquid influx is independent of air flowing out of control valve 36. Liquid can be flowing through control valve 36 or blocked, but humidified air is continuously free to flow out of humidifier tank 14. Control valve 36 can take many forms as described below with reference to FIGS. 4A-11.

FIG. 4A is a vertical cross sectional view, and FIG. 4B is a horizontal cross sectional view, of a first embodiment of control valve 36A. Control valve 36A includes top portion 62A, bottom portion 64A, liquid inlet port 66A, shut-off mechanism 68A, air vents 70A, neck 74A, shoulder 76A, body 78A, seal 80A, arm 82A having first end 84A and second end 86A, and float 88A. Control valve 36A defines air passageway 90A having inlet 92A and outlet 94A, and liquid passageway 96A having inlet 98A and outlet 100A. Humidified air can pass continuously through air passageway 90A while shut-off mechanism 68A controls flow of liquid out of liquid passageway 96A.

Control valve 36A of FIGS. 4A-4B is similar to control valve 36 of FIGS. 1-3, and like numerals indicate like components. Like control valve 36, control valve 36A is configured to be inserted into tank air outlet 30 of humidifier tank 14. Control valve 36A differs from control valve 36 in that liquid passageway 96A is placed to one side of air passageway 90A, and seal 80A is triangular. The similarities and differences between control valve 36A and control valve 36 are described in further detail below.

Upper portion 62A of control valve 36A can be further divided into neck 74A and shoulder 76A. Neck 74A is the topmost part of upper portion 62A, and has a bottom end attached to a top end of shoulder 76A. A bottom end of shoulder 76A is attached to a top end of body 78A. Neck 74A, shoulder 76A, and body 78A are substantially cylindrical and vertically stacked in series. Shoulder 76A has a largest relative diameter, neck 74A has a middle sized relative diameter, and body 78A has a smallest relative diameter. Body 78A extends vertically from upper portion 62A to lower portion 64A of control valve 36A. Located toward a center of body 78A is a plurality of oblong air vents 70A for receiving air into control valve 36A. A bottom end of body 78A is located at lower end 64A of control valve 36A and is attached to shut-off mechanism 68A. Shut-off mechanism 68A can be further divided into seal 80A, arm 82A, and float 88A. Seal 80A is a triangular protrusion attached to first end 84A of arm 82A. Arm 82A extends horizontally from body 78A at a lower portion 64A of control valve 36A. More specifically, arm 82A extends from first end 84A secured to the lower portion of body 78A to second end 86A spaced a horizontal distance away from first end 84A. In the depicted embodiment, arm 82A is substantially rectangular and substantially cylindrical float 88A extends upwardly form its attachment to second end 86A.

Control valve 36A defines substantially cylindrical air passageway 90A and substantially cylindrical liquid passageway 96A. Air passageway 90A is defined by one or more walls that extend vertically through body 78A, shoulder 76A, and neck 74A of control valve 36A. Inlet 92A of air passageway 90A is located on body 78A near air vents 70 toward lower end 64A of control valve 36A. Outlet 94A of air passageway 90A is located at a top end of neck 74A and is enlarged in comparison to the rest of air passageway 90A. Liquid passageway 96A is defined by one or more walls that extend through shoulder 76A and body 78A to shut-off mechanism 68A of control valve 36A. Inlet 98A of liquid passageway extends horizontally into shoulder 76A from liquid inlet port 66A at one side. Once within shoulder 76A, liquid passageway 96A makes an approximately right angled turn to extend vertically through one side of body 78A. Outlet 100A of liquid passageway 96A is located at lower portion 64A or bottom end of body 78A near shut-off mechanism 68A. Air passageway 90A is several times wider than liquid passageway 96A. As shown in FIG. 4B, liquid passageway 96A is located within air passageway 90A, but liquid passageway 96A is located off to one side of air passageway 90A.

Shut-off mechanism 68A is located at outlet 100A of liquid passageway 96A. When control valve 36A is placed in a humidifier tank where liquid levels are relatively high (see FIG. 3), float 88A will float on a top surface of the liquid. The flotation of float 88A causes arm 82A to assume a raised position such that it extends horizontally across outlet 100A of liquid passageway 96A. First end 84A of arm 82A will form an approximately right angle with liquid passageway 100A and push seal 80A upwardly into outlet 100A of liquid passageway to prevent liquid from exiting control valve 36A. Liquid will flow into inlet 98A and fill passageway 96A, but not exit outlet 100A due to seal formed by arm 82A and seal 80A. As liquid within the humidifier tank evaporates, float 88A will move downwardly along with a surface of depleted liquid. The lowered float 88A will cause arm 82A to drop and form an obtuse angle with liquid passageway 96A. Once arm 82A drops, it no longer forces seal 80A against outlet 100A so seal 80A can move downwardly out of its sealing arrangement with liquid passageway 96A. Liquid will be free to flow out of outlet 100A and into the humidifier tank to replenish the depleted liquid level. Accordingly, shut-off mechanism 68A responds to a level of liquid in humidifier tank by either blocking or allowing incoming liquid flow.

FIG. 5A is a vertical cross sectional view, and FIG. 5B is a horizontal cross sectional view, of a second embodiment of control valve 36B. Control valve 36B includes top portion 62B, bottom portion 64B, liquid inlet port 66B, shut-off mechanism 68B, air vents 70B, neck 74B, shoulder 76B, body 78B, seal 80B, arm 82B and float 88B. Control valve 36B defines air passageway 90B having inlet 92B and outlet 94B, and liquid passageway 96B having inlet 98B and outlet 100B. Humidified air can pass continuously through air passageway 90B while shut-off mechanism 68B controls flow of liquid out of liquid passageway 96B.

Control valve 36B of FIGS. 5A-5B is similar to control valve 36 of FIGS. 1-3 and control valve 36A of FIGS. 4A-4B, and like numerals indicate like components Like control valve 36, control valve 36B is configured to be inserted into tank air outlet 30 of humidifier tank 14. Control valve 36B differs from control valve 36A in that liquid passageway 96B is concentric with air passageway 90B, and arm 82B and float 88B are contained within lower portion 64B of control valve 36B. The similarities and differences between control valve 36B and control valves 36 and 36A are described in further detail below.

Upper portion 62B of control valve 36B can be further divided into neck 74B and shoulder 76B. Neck 74B is the topmost part of upper portion 62B, and has a bottom end attached to a top end of shoulder 76B. A bottom end of shoulder 76B is attached to a top end of body 78B. Neck 74B, shoulder 76B, and body 78B are substantially cylindrical and vertically stacked in series. Shoulder 76B has a largest relative diameter, neck 74B has a middle sized relative diameter, and body 78B has a smallest relative diameter. Body 78B extends vertically from upper portion 62B to lower portion 64B of control valve 36B. Located toward a center of body 78B is a plurality of air vents 70B for receiving air into control valve 36B. A bottom end of body 78B is located at lower end 64B of control valve 36B and is attached to shut-off mechanism 68B. Shut-off mechanism 68B can be further divided into seal 80B, arm 82B, and float 88B. Seal 80B is a triangular protrusion attached to a center of arm 82B. Arm 82B is substantially rectangular and located wholly within body 78B at lower portion 64B of control valve 36B. Located centrally within body 78B just below a center of arm 82B is spherical float 88B.

Control valve 36B defines substantially cylindrical air passageway 90B and substantially cylindrical liquid passageway 96B. Air passageway 90B is defined by one or more walls that extend vertically through body 78B, shoulder 76B, and neck 74B of control valve 36B. Inlet 92B of air passageway 90B is located on body 78B near air vents 70B toward lower end 64B of control valve 36B. Outlet 94B of air passageway 90B is located at a top end of neck 74B and is enlarged in comparison to the rest of air passageway 90B. Liquid passageway 96B is defined by one or more walls that extend through shoulder 76B and body 78B to shut-off mechanism 68B of control valve 36B. Inlet 98B of liquid passageway extends horizontally into shoulder 76B from liquid inlet port 66B at one side. Near a central location of shoulder 76B, liquid passageway 96B makes an approximately right angled turn to extend vertically through body 78B. Outlet 100B of liquid passageway 96B is located at lower portion 64B or bottom end of body 78B near shut-off mechanism 68B. Air passageway 90B is several times wider than liquid passageway 96B. As shown in FIG. 5B, a majority of liquid passageway 96B (the vertically extending portion in body 78B) is located concentrically within air passageway 90A.

Shut-off mechanism 68B is located at outlet 100B of liquid passageway 96B. When control valve 36B is placed in a humidifier tank where liquid levels are relatively high (see FIG. 3), float 88B will float on a top surface of the liquid. The flotation of float 88B pushes upwardly on arm 82A which lodges seal 80B into outlet 100B of liquid passageway 96B to prevent liquid from exiting control valve 36B. Liquid will flow into inlet 98B and fill passageway 96B, but not exit outlet 100B due to seal formed by float 88B, arm 82B, and seal 80B. As liquid within the humidifier tank vaporizes, float 88B will move downwardly along with a surface of depleted liquid. The lowered float 88B will allow arm 82B to drop away from outlet 100B of liquid passageway 96B. Once arm 82B drops, it no longer forces seal 80B against outlet 100B. Liquid will be free to flow out of outlet 100B and into the humidifier tank to replenish the depleted liquid level. Accordingly, shut-off mechanism 68B responds to a level of liquid in humidifier tank by either blocking or allowing incoming liquid flow.

FIG. 6A is a vertical cross sectional view, and FIG. 6B is a horizontal cross sectional view of a third embodiment of control valve 36C. Control valve 36C includes top portion 62C, bottom portion 64C, liquid inlet port 66C, shut-off mechanism 68C, air vents 70C, neck 74C, shoulder 76C, body 78C, seal 80C, arm 82C having first end 84C and second end 86C, and float 88C. Control valve 36C defines air passageway 90C having inlet 92C and outlet 94C, and liquid passageway 96C having inlet 98C and outlet 100C. Humidified air can pass continuously through air passageway 90C while shut-off mechanism 68C controls flow of liquid out of liquid passageway 96C.

Control valve 36C of FIGS. 6A-6B is similar to control valve 36 of FIGS. 1-3, control valve 36A of FIGS. 4A-4B, and control valve 36B of FIGS. 5A-5B, and like numerals indicate like components. Like control valve 36, control valve 36C is configured to be inserted into tank air outlet 30 of humidifier tank 14. Control valve 36C differs from control valve 36A in that outlet 100C of liquid passageway 96C is located on a side of body 78C and seal 80C is pin-shaped. The similarities and differences between control valve 36C and control valves 36, 36A, and 36B are described in further detail below.

Upper portion 62C of control valve 36C can be further divided into neck 74C and shoulder 76C. Neck 74C is the topmost part of upper portion 62C, and has a bottom end attached to a top end of shoulder 76C. A bottom end of shoulder 76C is attached to a top end of body 78C. Neck 74C, shoulder 76C, and body 78C are substantially cylindrical and vertically stacked in series. Shoulder 76C has a largest relative diameter, neck 74C has a middle sized relative diameter, and body 78C has a smallest relative diameter. Body 78C extends vertically from upper portion 62C to lower portion 64C of control valve 36C. Located toward a lower portion 64C are air vents 70C forming oblong openings into body 78C for receiving air into control valve 36C. A bottom end of body 78C is located at lower end 64C of control valve 36C and is attached to shut-off mechanism 68C. Shut-off mechanism 68C can be further divided into seal 80C, arm 82C, and float 88C. Seal 80C is pin-shaped having a tip end and opposite flat end, and is located within body 78C. The tip end of seal 80C is located near a center of body 78C, while the flat end of seal 80C is in contact with first end 84C of arm 82C. Arm 82C extends horizontally from body 78C at lower portion 64C of control valve 36C. More specifically, arm 82C extends from first end 84C secured to the lower portion of body 78C to second end 86C spaced a horizontal distance away from first end 84C. In the depicted embodiment, arm 82C is substantially rectangular and substantially cylindrical float 88C extends upwardly form its attachment to second end 86C.

Control valve 36C defines substantially cylindrical air passageway 90C and substantially cylindrical liquid passageway 96C. Air passageway 90C is defined by one or more walls that extend vertically through body 78C, shoulder 76C, and neck 74C of control valve 36C. Inlet 92C of air passageway 90C is located on body 78C near air vents 70C toward lower end 64C of control valve 36C. Outlet 94C of air passageway 90C is located at a top end of neck 74C and is enlarged in comparison to the rest of air passageway 90C. Liquid passageway 96C is defined by one or more walls that extend through shoulder 76C and body 78C. Inlet 98C of liquid passageway 96C extends horizontally into shoulder 76C from liquid inlet port 66C at one side of control valve 36C. Once within shoulder 76C, liquid passageway 96C makes an approximately right angled turn to extend vertically down one side of body 78C. Outlet 100C of liquid passageway 96C is an opening extending through body 78C and out of control valve 36C on the same side of body 78C as inlet 98C of liquid passageway 96C. In the depicted embodiment, outlet 100C is located centrally along body 78C above vents 70C and not far beneath liquid inlet port 66C. Air passageway 90C is several times wider than liquid passageway 96C. As shown in FIG. 6B, liquid passageway 96C is located to one side of air passageway 90C in a thickened portion of a wall defining body 78C.

When control valve 36C is placed in a humidifier tank where liquid levels are relatively high (see FIG. 3), float 88C will float on a top surface of the liquid. The flotation of float 88C causes arm 82C to assume a raised position such that it extends horizontally across the bottom end of body 78C at lower portion 64C of control valve 36C. Arm 82C will form an approximately right angle with body 78C and push upwardly on vertical pin seal 80C. More specifically, first end 84C of arm 82 will push upwardly on the flat end of seal 80C. This upward movement of seal 80C causes the pin end to slide past and block outlet 100C of liquid passageway to prevent liquid from exiting a side of control valve 36C. Liquid will flow into inlet 98C and fill passageway 96C, but not exit outlet 100C due to seal formed by arm 82C and seal 80C. As liquid within the humidifier tank vaporizes, float 88C will move downwardly along with a surface of depleted liquid. The lowered float 88C will cause arm 82C to drop and form an obtuse angle with liquid passageway 96C. Once arm 82C drops, it no longer forces the pin end of seal 80C past outlet 100C. Seal 80C moves downwardly out of its sealing arrangement with outlet 100C of liquid passageway 96A. Liquid will be free to flow out of outlet 100C and into the humidifier tank to replenish the depleted liquid level. Accordingly, shut-off mechanism 68C responds to a level of liquid in humidifier tank by either blocking or allowing incoming liquid flow.

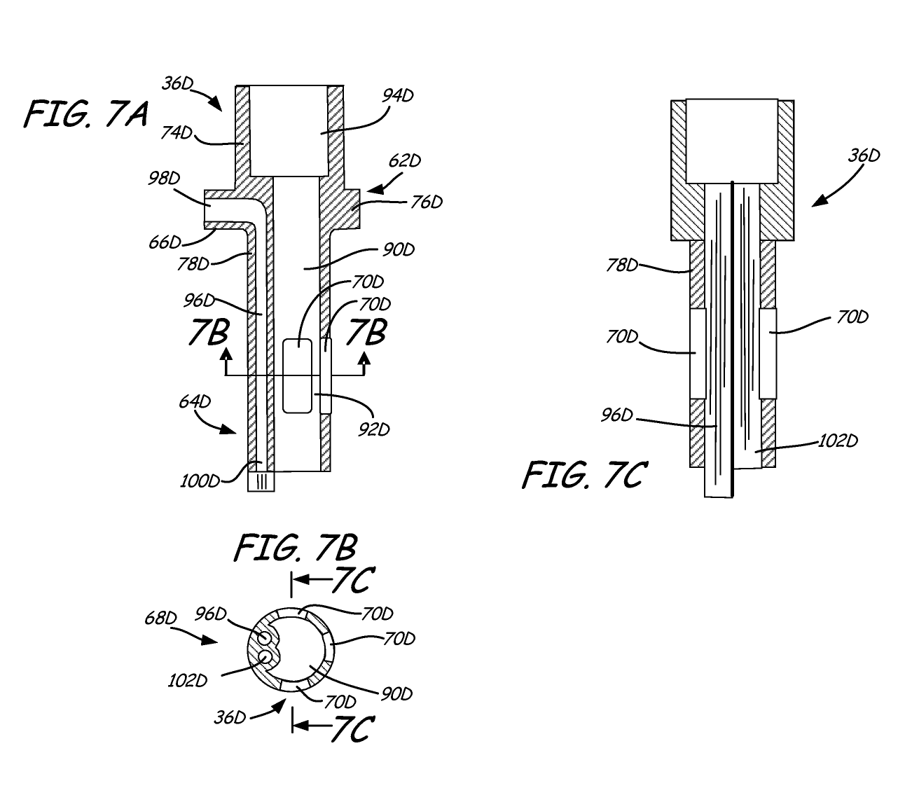

FIG. 7A is a vertical cross sectional view, FIG. 7B is a horizontal cross sectional view, and FIG. 7C is a vertical cross sectional view of a fourth embodiment of control valve 36D. Control valve 36D includes top portion 62D, bottom portion 64D, liquid inlet port 66D, shut-off mechanism 68D, air vents 70D, neck 74D, shoulder 76D, and body 78D. Control valve 36D defines air passageway 90D having inlet 92D and outlet 94D, liquid passageway 96D having inlet 98D and outlet 100D, and vacuum air vent 102D. Humidified air can pass continuously through air passageway 90D while shut-off mechanism 68D controls flow of liquid out of liquid passageway 96D.

Control valve 36D of FIGS. 7A-7C is similar to control valve 36 of FIGS. 1-3, control valve 36A of FIGS. 4A-4B, control valve 36B of FIGS. 5A-5B, and control valve 36C of FIGS. 6A-6B, and like numerals indicate like components. Like control valve 36, control valve 36D is configured to be inserted into tank air outlet 30 of humidifier tank 14. Control valve 36D differs from control valve 36 in that liquid passageway 96D and vacuum air vent 102D are placed to one side of air passageway 90A, and vacuum air vent 102 forms shut-off mechanism 68D. The similarities and differences between control valve 36D and control valves 36, 36A, 36B, and 36C are described in further detail below.

Upper portion 62D of control valve 36D can be further divided into neck 74D and shoulder 76D. Neck 74D is the topmost part of upper portion 62D, and has a bottom end attached to a top end of shoulder 76D. A bottom end of shoulder 76D is attached to a top end of body 78D. Neck 74D, shoulder 76D, and body 78D are substantially cylindrical and vertically stacked in series. Shoulder 76D has a largest relative diameter, neck 74D has a middle sized relative diameter, and body 78D has a smallest relative diameter. Body 78D extends vertically from upper portion 62D to lower portion 64D of control valve 36D. Located between a center of body 78D and lower portion 64D are a plurality of air vents 70D openings for receiving air into control valve 36D. A bottom end of body 78D is located at lower end 64D of control valve 36D

Control valve 36D defines substantially cylindrical air passageway 90D, substantially cylindrical liquid passageway 96D, and substantially cylindrical vacuum air vent 102D. Air passageway 90D is defined by one or more walls that extend vertically through body 78D, shoulder 76D, and neck 74D of control valve 36D. Inlet 92D of air passageway 90D is located on body 78D near air vents 70D toward lower end 64D of control valve 36D. Outlet 94D of air passageway 90D is located at a top end of neck 74D and is enlarged in comparison to the rest of air passageway 90D. Liquid passageway 96D is defined by one or more walls that extend through shoulder 76D and body 78D to lower end 36D control valve 36D. Inlet 98D of liquid passageway extends horizontally into shoulder 76D from liquid inlet port 66D at one side of control valve 36D. Once within shoulder 76D, liquid passageway 96D makes an approximately right angled turn to extend vertically downward through one side of body 78D. Outlet 100D of liquid passageway 96D is located at lower portion 64D or bottom end of body 78D. Vacuum air vent 102D is located next to liquid passageway 96D and follows the same trajectory through control valve 36D. Air passageway 90D is several times wider than liquid passageway 96D. As shown in FIG. 4B, liquid passageway 96D is located to one side of air passageway 90D and is adjacent vacuum air vent 102D.

Extending along with liquid passageway 96D is air vent 102. Like liquid passageway 96D, vacuum air vent 102D is defined by one or more walls that extend through shoulder 76D and body 78D to lower end 36D control valve 36D. An inlet of vacuum air vent 102D is located at lower portion 64D or bottom end of body 78D right next to outlet 100D of liquid passageway 96D. Vacuum air vent 102D extends vertically upward through one side of body 78D alongside liquid passageway 96D. Once within shoulder 76D, vacuum air vent 102D makes an approximately right angled turn to its outlet located at liquid inlet port 66D adjacent inlet 98D of liquid passageway 96D. In the depicted embodiment, shut-off mechanism 68D is vacuum air vent 102D, which functions as a vacuum seal to regulate the flow of liquid through control valve 36D.

To use control valve 36D, a liquid conduit (such as liquid conduit 38 shown in FIG. 1) should additionally include an air conduit. The dual fluid conduit is attached to liquid inlet port 66D and provides liquid to liquid passage 96D while simultaneously providing for air to escape out of vacuum air vent 102D. When control valve 36D is placed in a humidifier tank where liquid levels are relatively high (see FIG. 3), a top surface of liquid will touch both outlet 100D of liquid passageway 39D and outlet of vacuum air vent 102D. Since air cannot escape from the humidifier tank through vacuum air vent 102D, liquid is prevented from exiting outlet 100D of liquid passageway 96D. As liquid within the humidifier tank vaporizes, a top surface of liquid will no longer be in contact with outlet 100D of liquid passageway 96D and the inlet of vacuum air vent 102D. Since air can escape from tank through vacuum air vent 102D, liquid will be free to flow out of outlet 100D and into the humidifier tank to replenish the depleted liquid level. Accordingly, vacuum air vent 102D functions as shut-off mechanism 68D that responds to a level of liquid in humidifier tank by either blocking or allowing incoming liquid flow.

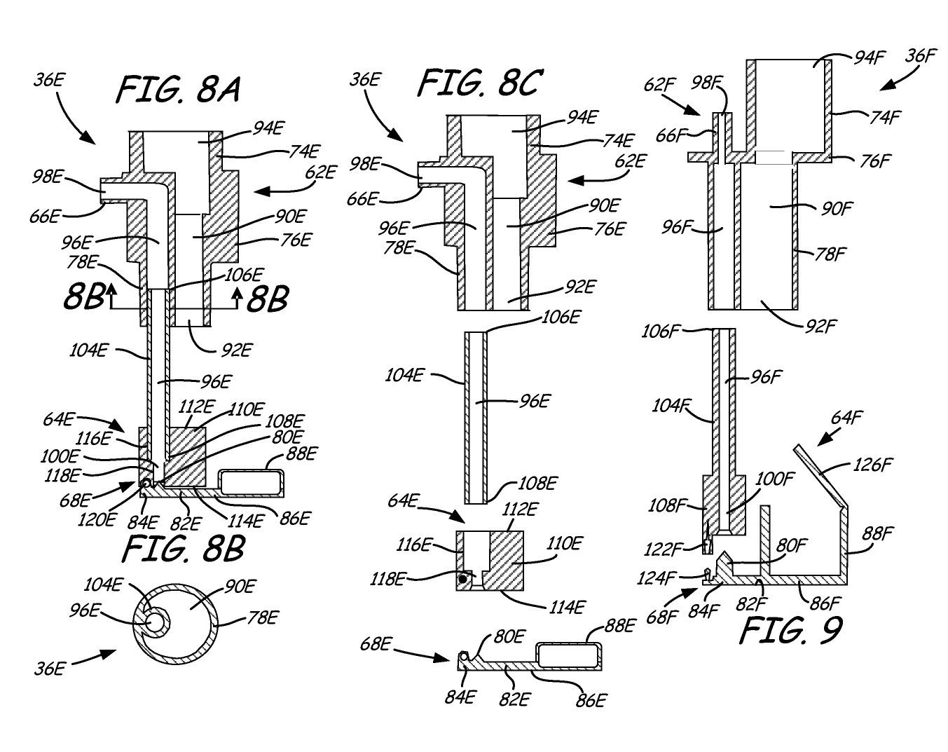

FIG. 8A is a vertical cross sectional view, FIG. 8B is a horizontal cross sectional view, and FIG. 8C is an exploded vertical cross sectional view, of a fifth embodiment of control valve 36E. Control valve 36E includes top portion 62E, bottom portion 64E, liquid inlet port 66E, shut-off mechanism 68E, neck 74E, shoulder 76E, body 78E, seal 80E, arm 82E having first end 84E and second end 86E, float 88E. Control valve 36E defines air passageway 90E having inlet 92E and outlet 94E, and liquid passageway 96E having inlet 98E and outlet 100E. Control valve 36E further includes tube 104E having first end 106E and second end 108E, and valve block 110E having top 112E, bottom 114E, first cavity 116E, second cavity 118E, and pin 120E. Humidified air can pass continuously through air passageway 90E while shut-off mechanism 68E controls flow of liquid out of liquid passageway 96E.

Control valve 36E of FIGS. 8A-8C is similar to control valve 36 of FIGS. 1-3, control valve 36A of FIGS. 4A-4B, control valve 36B of FIGS. 5A-5B, control valve 36C of FIGS. 6A-6B, and control valve 36D of FIGS. 7A-7C, and like numerals indicate like components. Like control valve 36, control valve 36E is configured to be inserted into tank air outlet 30 of humidifier tank 14. Control valve 36E differs from control valve 36 in that valve 36E is formed in discrete pieces, tube 104E connects upper portion 62E to lower portion 64E, and seal 80E is triangular. The similarities and differences between control valve 36E and control valves 36, 36A, 36B, 36C, and 36D are described in further detail below.

Upper portion 62E of control valve 36E can be further divided into neck 74E and shoulder 76E. Neck 74E is the topmost part of upper portion 62E, and has a bottom end attached to a top end of shoulder 76E. A bottom end of shoulder 76E is attached to a top end of body 78E. Neck 74E, shoulder 76E, and body 78E are substantially cylindrical and vertically stacked in series. Shoulder 76E has a largest relative diameter, neck 74E has a middle sized relative diameter, and body 78E has a smallest relative diameter. Body 78E extends vertically downwards from shoulder 76E a relatively short distance. Lower end 64E of control valve 36E is spaced a distance from lower end of body 78E by tube 104E. A first end 106E of tube 104E extends into a lower end of body 78E and a second end 108E of tube 104E is attached to valve block 110E. Second end 108E of tube 104E extends through top 112E and into first cavity 116E of valve block 110E. Shut-off mechanism 68E, including seal 80E, arm 82E and float 88E, is attached to bottom 114E of valve block 110E. Seal 80E is a triangular protrusion attached to first end 84E of arm 82E. Seal 80E extends upwardly into second cavity 118E on bottom 114E of valve block 110E. Arm 82E extends horizontally from bottom 114E of valve block 110E at lower portion 64E of control valve 36E. More specifically, arm 82E extends from first end 84E secured to the bottom 114 of valve block 110 to second end 86E spaced a horizontal distance away from first end 84E. In the depicted embodiment, arm 82E is substantially rectangular and substantially cylindrical float 88E extends upwardly form its attachment to second end 86E.

Control valve 36E defines substantially cylindrical air passageway 90E and substantially cylindrical liquid passageway 96E. Air passageway 90E is defined by one or more walls that extend vertically through body 78E, shoulder 76E, and neck 74E of control valve 36E. Inlet 92E of air passageway 90E is located at a bottom end of body 78E. Outlet 94E of air passageway 90E is located at a top end of neck 74E and is enlarged in comparison to the rest of air passageway 90E. Liquid passageway 96E is defined by one or more walls that extend through shoulder 76E and body 78E, as well as by tube 104E, and first and second cavities 114E, 116E extending through valve block 110E. Inlet 98E of liquid passageway extends horizontally into shoulder 76E from liquid inlet port 66E at one side. Once within shoulder 76E, liquid passageway 96E makes an approximately right angled turn to extend vertically through one side of body 78E. Liquid passageway 96E continues from body 78E, through the length of tube 104E and through first cavity 116E and second cavity 118E of valve block 110E. Outlet 100E of liquid passageway 96E is located at second cavity 118E of valve block 110E near bottom 114E and shut-off mechanism 68E. Air passageway 90E is several times wider than liquid passageway 96E, but liquid passageway 96E is about twice as long as air passageway 96E. As shown in FIG. 8B, liquid passageway 96E is located next to air passageway 90E with liquid passageway 96E being located to one side of air passageway 90E.

Control valve 36E is assembled by inserting first end 106E of tube 104E into liquid passageway 96E defined in body 78E, and by inserting second end 108E of tube 104E into liquid passageway 96E defined in first cavity 116E of valve block 110E. Shut-off mechanism 68E is secured to bottom 114E of valve block 110E by snapping first end 84E or arm 82E into attachment with valve block 110E or by passing pin 120E through openings in both arm 82E and valve block 110E. Shut-off mechanism 68E is located at outlet 100E of liquid passageway 96E.

When control valve 36E is placed in a humidifier tank where liquid levels are relatively high (see FIG. 3), float 88E will float on a top surface of the liquid. The flotation of float 88E causes arm 82E to assume a raised portion such that it extends horizontally across outlet 100E of liquid passageway 96E. First end 84E of arm 82E will form an approximately right angle with liquid passageway 100E and push seal 80E upwardly into outlet 100E of liquid passageway to prevent liquid from exiting control valve 36E. Liquid will flow into inlet 98E and fill passageway 96E, but not exit outlet 100E due to seal formed by arm 82E and seal 80E. As liquid within the humidifier tank evaporates, float 88E will move downwardly along with a surface of depleted liquid. The lowered float 88E will cause arm 82E to drop and form an obtuse angle with liquid passageway 96E. Once arm 82E drops, it no longer forces seal 80E against outlet 100E. Seal 80E moves downwardly out of its sealing arrangement with liquid passageway 96E. Liquid will be free to flow out of outlet 100E and into the humidifier tank to replenish the depleted liquid level. Accordingly, shut-off mechanism 68E responds to a level of liquid in humidifier tank by either blocking or allowing incoming liquid flow.

FIG. 9 is an exploded cross sectional view of a sixth embodiment of control valve 36F. Control valve 36F includes top portion 62F, bottom portion 64F, liquid inlet port 66F, shut-off mechanism 68F, neck 74F, shoulder 76F, body 78F, seal 80F, arm 82F having first end 84F and second end 86F, float 88F. Control valve 36F defines air passageway 90F having inlet 92F and outlet 94F, and liquid passageway 96F having inlet 98F and outlet 100F. Control valve 36F further includes tube 104F having first end 106F and second end 108F, pin cavity 122F, pin 124F, and flap 126F. Humidified air can pass continuously through air passageway 90F while shut-off mechanism 68F controls flow of liquid out of liquid passageway 96F.

Control valve 36F of FIG. 9 is similar to control valve 36 of FIGS. 1-3, control valve 36A of FIGS. 4A-4B, control valve 36B of FIGS. 5A-5B, control valve 36C of FIGS. 6A-6B, control valve 36D of FIGS. 7A-7C, and control valve 36E of FIGS. 8A-8C, and like numerals indicate like components. Like control valve 36, control valve 36F is configured to be inserted into tank air outlet 30 of humidifier tank 14. Control valve 36F differs from control valve 36 in that valve 36F is formed in discrete pieces, tube 104F connects upper portion 62F to lower portion 64F, seal 80F is triangular, and float 88F includes flap 126F. The similarities and differences between control valve 36F and control valves 36, 36A, 36B, 36C, 36D, and 36E are described in further detail below.

Neck 74F is the topmost part of upper portion 62F and extends vertically upward from shoulder 76F. Liquid inlet port 66F also extends vertically upward from shoulder 76F parallel to neck 74F. Shoulder 76F extends horizontally between a bottom of neck 74F and liquid inlet port 66F and a top of body 78F. Neck 74F and body 78F are substantially cylindrical and vertically stacked, but neck 74F is offset to one side of body 78F. Body 78F extends vertically downwards from neck 74F and shoulder 76F a relatively short distance. Lower end 64F of control valve 36F is spaced a distance from lower end of body 78F by tube 104F. A first end 106F of tube 104F extends into a lower end of body 78F, and a second end 108F of tube 104F is attached to shut-off mechanism 68F. Pin hole 122F extends upwardly into second end 108F of tube 104F, and pin 124F extends upwardly from first end 84F of arm 82F. Pin 124F is snapped into pin hole 122F to attach arm 82F to tube 104F. Seal 80F is a triangular protrusion attached to first end 84F of arm 82F next to pin 124F. Seal 80F extends upwardly into second end 108F of tube 104F. Arm 82F extends horizontally from first end 84F to second end 86F spaced a horizontal distance away from first end 84F. In the depicted embodiment, arm 82F is substantially rectangular and substantially rectangular float 88F extends upwardly form its attachment to second end 86F. In the depicted embodiment, float 88F is molded as an open structure and then assembled into a box by moving flap 126F to snap into place as a top that defines hollow internal space of float 88F.

Control valve 36F defines substantially cylindrical air passageway 90F and substantially cylindrical liquid passageway 96F. Air passageway 90F is defined by one or more walls that extend vertically through body 78F, shoulder 76F, and neck 74F of control valve 36F. Inlet 92F of air passageway 90F is located at a bottom end of body 78F. Outlet 94F of air passageway 90F is located at a top end of neck 74F and is enlarged in comparison to the rest of air passageway 90F. Liquid passageway 96F is defined by one or more walls that extend through shoulder 76F and body 78F, as well as by tube 104F. Inlet 98F of liquid passageway 96F extends vertically downward from liquid inlet port 66F through shoulder 76F and through one side of body 78F. From body 78F, liquid passageway 96F continues the length of tube 104F. Outlet 100F of liquid passageway 96F is located second end 108F of tube 104F. Air passageway 90F is wider than liquid passageway 96F, and liquid passageway 90F is longer than air passageway 90F.

Control valve 36F is assembled by inserting first end 106F of tube 104F into liquid passageway 96F defined in body 78F. Shut-off mechanism 68E is secured to tube 104F by snapping pin 124F into its mating relationship with pin hole 122F. So assembled, shut-off mechanism 68F is located at outlet 100F of liquid passageway 96F. When control valve 36F is placed in a humidifier tank where liquid levels are relatively high (see FIG. 3), float 88F will float on a top surface of the liquid. The flotation of float 88F causes arm 82F to assume a raised position such that it extends horizontally across outlet 100F of liquid passageway 96F. First end 84F of arm 82F will form an approximately right angle with liquid passageway 100F and push seal 80F upwardly into outlet 100F of liquid passageway 96F to prevent liquid from exiting control valve 36F. Liquid will flow into inlet 98F and fill passageway 96F, but not exit outlet 100F due to seal formed by arm 82F and seal 80F. As liquid within the humidifier tank evaporates, float 88F will move downwardly along with a surface of depleted liquid. The lowered float 88F will cause arm 82F to drop and form an obtuse angle with liquid passageway 96F. Once arm 82F drops, it no longer forces seal 80F against outlet 100F. Seal 80F moves downwardly out of its sealing arrangement with liquid passageway 96F. Liquid will be free to flow out of outlet 100F and into the humidifier tank to replenish the depleted liquid level. Accordingly, shut-off mechanism 68F responds to a level of liquid in humidifier tank by either blocking or allowing incoming liquid flow.

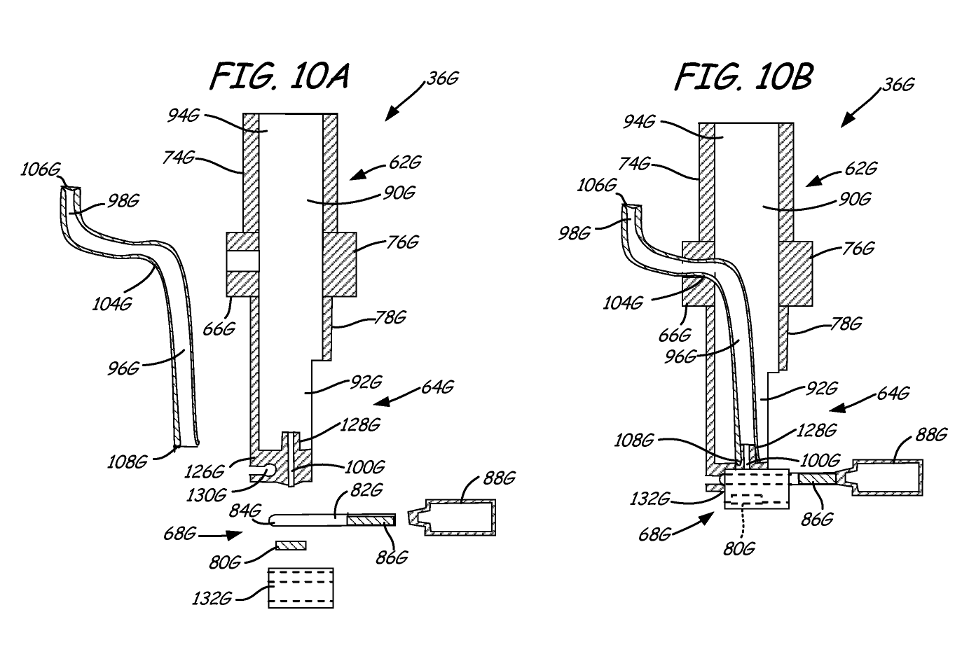

FIG. 10A is an exploded cross sectional view, and FIG. 10B is an assembled cross sectional view, of a seventh embodiment of control valve 36G. Control valve 36G includes top portion 62G, bottom portion 64G, liquid inlet port 66G, shut-off mechanism 68G, neck 74G, shoulder 76G, body 78G, seal 80G, arm 82G having first end 84G and second end 86G, float 88G. Control valve 36G defines air passageway 90G having inlet 92G and outlet 94G, and liquid passageway 96G having inlet 98G and outlet 100G. Control valve 36G further includes tube 104G having first end 106G and second end 108G, and flange 126G, nipple 128G, cavity 130G, and seal housing 132G. Humidified air can pass continuously through air passageway 90G while shut-off mechanism 68G controls flow of liquid out of liquid passageway 96G.

Control valve 36G of FIGS. 10A-10B is similar to control valve 36 of FIGS. 1-3, control valve 36A of FIGS. 4A-4B, control valve 36B of FIGS. 5A-5B, control valve 36C of FIGS. 6A-6B, control valve 36D of FIGS. 7A-7C, control valve 36E of FIGS. 8A-8C, and control valve 36F of FIG. 9, and like numerals indicate like components. Like control valve 36, control valve 36G is configured to be inserted into tank air outlet 30 of humidifier tank 14. Control valve 36G differs from control valve 36 in that valve 36G is formed in discrete pieces, tube 104G extends through liquid inlet port 66G, and through body 78G to shut-off mechanism 68G, and valve 80G is rectangular. The similarities and differences between control valve 36G and control valves 36, 36A, 36B, 36C, 36D, 36E, and 36F are described in further detail below.

Upper portion 62G of control valve 36G can be further divided into neck 74G and shoulder 76G. Neck 74G is the topmost part of upper portion 62G, and has a bottom end attached to a top end of shoulder 76G. A bottom end of shoulder 76G is attached to a top end of body 78G. Liquid inlet port 66G is an opening extending horizontally into shoulder 76G. Neck 74G, shoulder 76G, and body 78EG are substantially cylindrical and vertically stacked in series. When viewed in vertical cross section, neck 74G, shoulder 76G, and body 78G are rectangular in shape. Shoulder 76G has a largest relative diameter, neck 74G has a middle sized relative diameter, and body 78G has a smallest relative diameter. Body 78G extends vertically downwards from shoulder 76G to lower end 64G of control valve 36G. One vertical side of body 78G is closed, but an opening extends into the opposite vertical side of body 78G for receiving air.

Tube 104G is a flexible conduit that extends through control valve 36G from liquid inlet port 66G to shut off mechanism 68G. A first end 106G of tube 104G extends horizontally through liquid inlet port 66G. Once through shoulder 76G, tube 104G curves vertically downward to extend through body 78G. Second end 108G of tube 104G cooperates with shutoff mechanism 68G at lower end 64G of control valve 36G. Bottom end of body 78G has horizontally extending flange 126G. Nipple 128G extends vertically upwards from a center of flange 126G for receiving second end 108G of tube 104G. Cavity 130G extends into a side of flange 126G for coupling with arm 82G. Shut-off mechanism 68G, including seal 80G, arm 82G and float 88G, is attached to flange 130G at a bottom end of body 78G. Seal 80G is substantially rectangular and housed within a bottom end of a substantially rectangular seal housing 132G. First end 84G of arm 84G cooperates with cavity 130G to attach arm 82G to flange 126G. First end 84G of arm 82G extends horizontally through an upper end of seal housing 132G. Second end 86G of arm 82G is attached to float 88G. Accordingly, arm 82G extends from first end 84G to second end 86G spaced a horizontal distance away from first end 84G. In the depicted embodiment, arm 82G is substantially cylindrical and substantially rectangular float 88G extends form its attachment to second end 86G.

Control valve 36G defines substantially cylindrical air passageway 90G and substantially cylindrical liquid passageway 96G. Air passageway 90G is defined by one or more walls that extend vertically through body 78G, shoulder 76G, and neck 74G of control valve 36G. Inlet 92G of air passageway 90G is the opening located at a bottom end of body 78G. Outlet 94G of air passageway 90G is located at a top end of neck 74G. Liquid passageway 96G is defined mostly by tube 104G. Inlet 98G of liquid passageway 98G extends horizontally into shoulder 76G from liquid inlet port 66G at one side. Once within shoulder 76G, liquid passageway 98G makes an approximately right angled turn to extend vertically through body 78G. Liquid passageway 98G continues the length of tube 104G to second end 108G, which is attached to nipple 128G. Liquid passageway 98G extends through a center of nipple 128G to outlet 100G located at bottom end of flange 126G. Outlet 100G of liquid passageway 96G is located at shut-off mechanism 68G. Air passageway 90G is several times wider than liquid passageway 96G. As shown in FIG. 10B, liquid passageway 96G is located within air passageway 90G when both are extending through body 78G.

Control valve 36G is assembled by inserting second end 108G of tube 104G through liquid inlet port 66G and body 78G. Second end 108G of tube 104G is attached to nipple 128G on flange 108G, and first end 106G of tube 104G extends out of liquid inlet port 66G. Shut-off mechanism 68G is secured to body 78G by snapping first end 84G or arm 82G into attachment with opening 130G in flange 126G. Seal 80G is placed the bottom end of seal housing 132G, and first end 84G of arm 82G extends through the upper end of seal housing 132G. Float 88G is secured to second end 86G of arm 82G. So assembled, shut-off mechanism 68G is located at outlet 100G of liquid passageway 96G.

When control valve 36G is placed in a humidifier tank where liquid levels are relatively high (see FIG. 3), float 88G will float on a top surface of the liquid. The flotation of float 88G causes arm 82G to assume a raised portion such that it extends horizontally across outlet 100G of liquid passageway 96G. First end 84G of arm 82G will form an approximately right angle with liquid passageway 100G and pull seal housing 132G including seal 80G upwardly toward outlet 100G of liquid passageway to prevent liquid from exiting control valve 36G. Liquid will flow into inlet 98G and fill passageway 96G/tube 104G, but not exit outlet 100G due to seal formed by arm 82G and seal 80G. As liquid within the humidifier tank evaporates, float 88G will move downwardly along with a surface of depleted liquid. The lowered float 88G will cause arm 82G to drop and form an obtuse angle with liquid passageway 96G. Once arm 82G drops, it no longer forces seal 80G against outlet 100G. Seal 80G moves downwardly out of its sealing arrangement with liquid passageway 96G. Liquid will be free to flow out of outlet 100G and into the humidifier tank to replenish the depleted liquid level. Accordingly, shut-off mechanism 68G responds to a level of liquid in humidifier tank by either blocking or allowing incoming liquid flow.

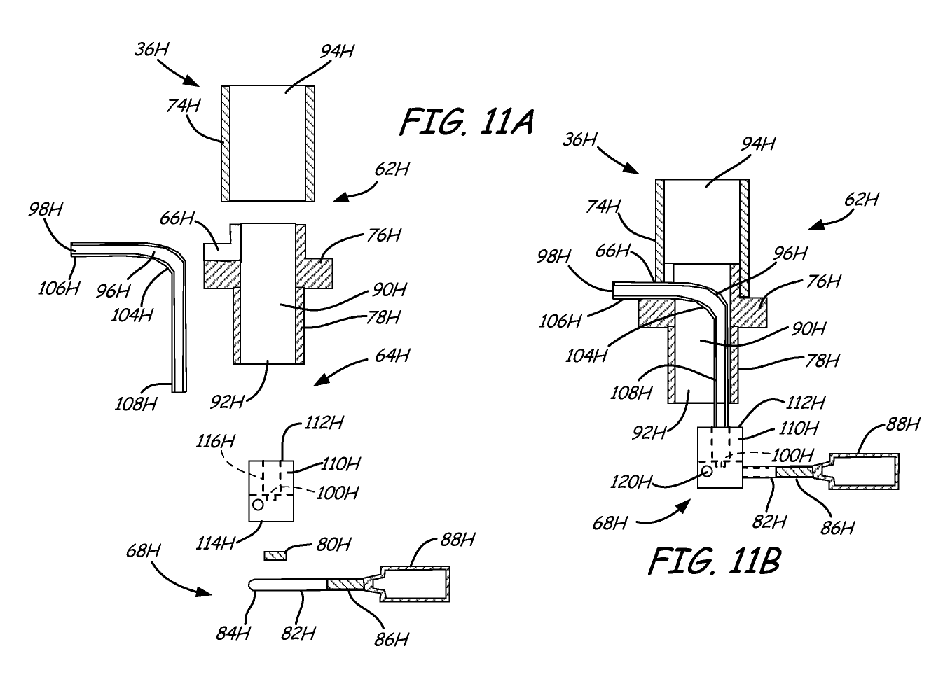

FIG. 11A is an exploded cross sectional view, and FIG. 11B is an assembled cross sectional view, of an eighth embodiment of control valve 36H. Control valve 36H includes top portion 62H, bottom portion 64H, liquid inlet port 66H, shut-off mechanism 68H, neck 74H, shoulder 76H, body 78H, seal 80H, arm 82H having first end 84H and second end 86H, and float 88H. Control valve 36H defines air passageway 90H having inlet 92H and outlet 94H, and liquid passageway 96H having inlet 98H and outlet 100H. Control valve 36H further includes tube 104H having first end 106H and second end 108H, and valve block 110H having top 112H, bottom 114H, cavity 116H, and pin 120H. Humidified air can pass continuously through air passageway 90H while shut-off mechanism 68H controls flow of liquid out of liquid passageway 96H.

Control valve 36H of FIGS. 11A-11B is similar to control valve 36 of FIGS. 1-3, control valve 36A of FIGS. 4A-4B, control valve 36B of FIGS. 5A-5B, control valve 36C of FIGS. 6A-6B, control valve 36D of FIGS. 7A-7C, control valve 36E of FIGS. 8A-8C, control valve 36F of FIG. 9, and control valve 36G of 10A-10B, and like numerals indicate like components. Like control valve 36, control valve 36H is configured to be inserted into tank air outlet 30 of humidifier tank 14. Control valve 36H differs from control valve 36 in that valve 36H is formed in discrete pieces, tube 104H extends through liquid inlet port 66H, and through body 78H to shut-off mechanism 68H, and valve 80H is rectangular. The similarities and differences between control valve 36H and control valves 36, 36A, 36B, 36C, 36D, 36E, 36F, and 36G are described in further detail below.

Upper portion 62H of control valve 36H can be further divided into neck 74H and shoulder 76H. Neck 74H is the topmost part of upper portion 62H, and has a bottom end that receives a top end of shoulder 76H. A bottom end of shoulder 76H is attached to a top end of body 78H. Shoulder 76H and body 78H can be integrally formed. Liquid inlet port 66H is an opening or window extending into one side of shoulder 76H. Neck 74H, shoulder 76H, and body 78EH are substantially cylindrical and vertically stacked in series. Shoulder 76H has a largest relative diameter, neck 74H has a middle sized relative diameter, and body 78H has a smallest relative diameter. Body 78H extends vertically downwards from shoulder 76H to lower end 64H of control valve 36H. The bottom end of body 78G is open for receiving air.

Tube 104H is a rigid conduit that extends through control valve 36H from liquid inlet port 66H to shut off mechanism 68H. First end 106H of tube 104H extends horizontally through liquid inlet port 66H, and is trapped between a top end of shoulder 78H and a bottom end of neck 74H. Once through shoulder 76H, tube 104H curves vertically downward to extend through body 78H. Second end 108H of tube 104H cooperates with shutoff mechanism 68H at lower end 64H of control valve 36H. Shut-off mechanism 68H, including seal 80H, arm 82H and float 88H, is attached to valve block 110H, which is suspending from tube 104H. Second end 108H of tube 104H extends through top 114H and into cavity 116H of valve block 110H. Seal 80H is substantially rectangular and housed within valve block 110H near bottom 114H. Arm 82H extends horizontally from valve block 110H at lower portion 64H of control valve 36H. More specifically, arm 82H extends from first end 84H secured to the bottom 114H of valve block 110H by pin 120H, to second end 86H spaced a horizontal distance away from first end 84H. In the depicted embodiment, arm 82H is substantially cylindrical and substantially rectangular float 88H is attached to second end 86J.

Control valve 36H defines substantially cylindrical air passageway 90H and substantially cylindrical liquid passageway 96H. Air passageway 90H is defined by one or more walls that extend vertically through body 78H, shoulder 76H, and neck 74H of control valve 36H. Inlet 92H of air passageway 90H is the opening located at a bottom end of body 78H. Outlet 94H of air passageway 90H is located at a top end of neck 74H. Liquid passageway 96H is defined mostly by tube 104H. Inlet 98H of liquid passageway 98H extends horizontally into shoulder 76H from liquid inlet port 66H at one side. Once within shoulder 76H, liquid passageway 98H makes an approximately right angled turn to extend vertically through body 78H. Liquid passageway 98H continues the length of tube 104H to second end 108H, which is attached to cavity 116H of valve body 110H. Liquid passageway 98H extends through cavity 116H to outlet 100H located in valve body 110H. Outlet 100H of liquid passageway 96H is located at shut-off mechanism 68H (seal 80H and arm 82H). Air passageway 90H is several times wider than liquid passageway 96H. As shown in FIG. 11B, liquid passageway 96H is located within air passageway 90H when both are extending through body 78H.

Control valve 36H is assembled by inserting second end 108H of tube 104H through liquid inlet port 66H and body 78H. Top end of shoulder 76H is then inserted into the bottom end of neck 74H, thereby trapping first end 98H of tube 104H between a bottom end of neck 74H and a top end of shoulder 76H at liquid inlet port 66H. First end 98H of tube 104H should extend horizontally out of liquid inlet port 66H. Second end 108H of tube 104H is secured to shut-off mechanism 68H. Shut-off mechanism 68H is secured to body 78H by inserting second end 108H of tube 104H into cavity 116H of valve body 110H. Seal 80G is inserted into valve block 110H near liquid outlet 100H. First end 84H of arm 82H is secured to valve body 110H by pin 120H or other suitable means of attachment. Float 88H is secured to second end 86H of arm 82H. So assembled, arm 82H and seal 80H form shut-off mechanism 68H located at outlet 100H of liquid passageway 96H.

When control valve 36H is placed in a humidifier tank where liquid levels are relatively high (see FIG. 3), float 88H will float on a top surface of the liquid. The flotation of float 88H causes arm 82H to assume a raised portion such that it extends horizontally across outlet 100H of liquid passageway 96H. First end 84H of arm 82H will form an approximately right angle with liquid passageway 96H and pull seal 80H upwardly toward outlet 100H of liquid passageway 96H to prevent liquid from exiting control valve 36H. Liquid will flow into inlet 98H and fill passageway 96H/tube 104H, but not exit outlet 100H due to seal formed by arm 82H and seal 80H. As liquid within the humidifier tank evaporates, float 88H will move downwardly along with a surface of depleted liquid. The lowered float 88H will cause arm 82H to drop and form an obtuse angle with liquid passageway 96H. Once arm 82H drops, it no longer forces seal 80H against outlet 100H. Seal 80H moves downwardly out of its sealing arrangement with liquid passageway 96H. Liquid will be free to flow out of outlet 100H and into the humidifier tank to replenish the depleted liquid level. Accordingly, shut-off mechanism 68H responds to a level of liquid in humidifier tank by either blocking or allowing incoming liquid flow.

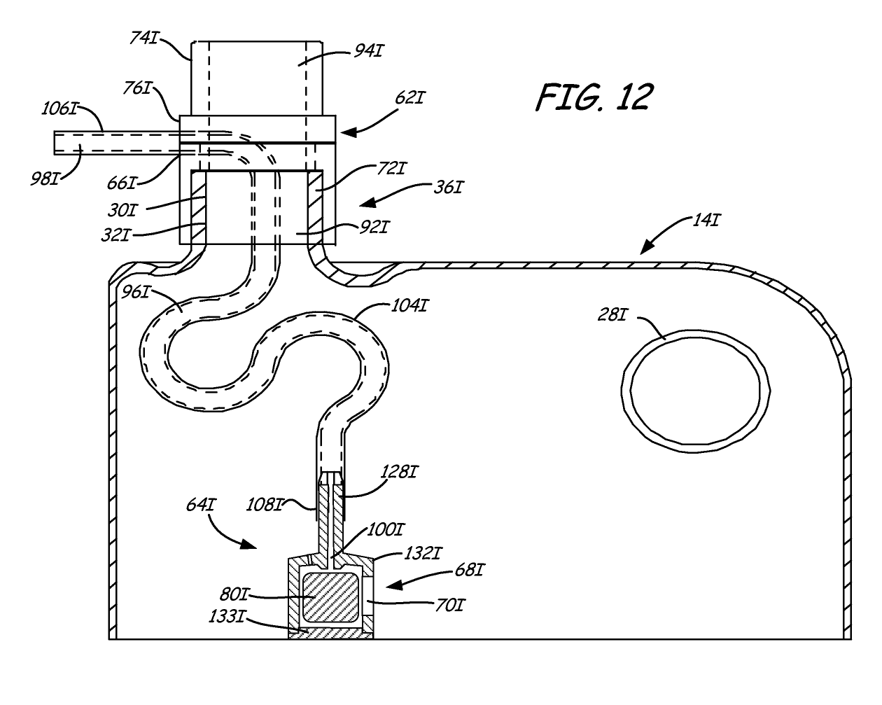

FIG. 12 a vertical cross sectional view of humidifier tank 14I having drop in control valve 36I. Humidifier tank 14I includes tank air inlet 28I and collar 72I. Control valve 36I includes top portion 62I, bottom portion 64I, liquid inlet port 66I, shut-off mechanism 68I, neck 74I, shoulder 76I, seal 80I, air passageway 90I having inlet 92I and outlet 94I, and liquid passageway 96I having inlet 98I and outlet 100I, tube 104I having first end 106I and second end 108I, nipple 128I, seal housing 132I, and retainer 133I. Control valve 36I simultaneously allows a flow of humidified air to through air passageway 90I and a flow of liquid through liquid passageway 96I.

Control valve 361 of FIG. 12 is similar to control valve 36 of FIGS. 1-3, control valve 36A of FIGS. 4A-4B, control valve 36B of FIGS. 5A-5B, control valve 36C of FIGS. 6A-6B, control valve 36D of FIGS. 7A-7C, control valve 36E of FIGS. 8A-8C, control valve 36F of FIG. 9, control valve 36G of FIGS. 10A-10B, and control valve 36H of FIGS. 11A-11B, and like numerals indicate like components. Like control valve 36, control valve 36I is configured to be inserted into tank air outlet 30 of humidifier tank 14. Control valve 36I differs from control valve 36 in that valve 36I lacks body 78, tube 104I extends through liquid inlet port 66I to shut-off mechanism 681, and float 80I doubles as a seal. The similarities and differences between control valve 36I and control valves 36, 36A, 36B, 36C, 36D, 36E, 36F, 36G, and 36H are described in further detail below.

Humidifier tank 14I includes a vertically raised collar 72I, which defines a common, circular, opening for both tank air outlet 30I and tank liquid inlet 32I. Upper portion 62I of control valve 36I extends around collar 72I such with some of upper portion 62I located vertically above collar 72I. Upper portion 62I of control valve 36I can be further divided into neck 74I and shoulder 76I. Neck 74I, shoulder 76I are substantially cylindrical and vertically stacked in series. Neck 74I is the topmost portion of upper portion 62I and is sized to mate with (e.g. fit inside of) a humidifier conduit (such as first end 50 of humidifier conduit 48 shown in FIG. 2). Shoulder 76I has a top end attached to neck 74I, and a bottom end surrounding collar 72I. Shoulder 76I has a diameter larger than neck 74I and collar 72I and is configured to fit over collar 72I. An outside surface of collar 72I and an inside surface of neck 74I can form a light interference fit. Liquid inlet port 66I extends into a side of shoulder 76I.

Tube 104I is a flexible conduit that extends through control valve 36I from liquid inlet port 66I to shut off mechanism 68I. First end 106I of tube 104I extends horizontally through liquid inlet port 66I. Once through shoulder 76I, tube 104I curves vertically downward through the bottom open end of shoulder 76I and into tank 14I. Second end 108I of tube 104I cooperates with shutoff mechanism 68I at lower end 64I of control valve 36I. Nipple 128I extends vertically upwards from a valve housing 132I to receive second end 108GI of tube 104GI. In the embodiment depicted, second end 108I of tube 104I extends over nipple 128I thereby securing tube 104I to shut-off mechanism 64I. Opening or window 701 extends into a side of seal housing 132I to allow fluid follow into seal housing 132I. Seal housing 132I has an interior cavity for housing seal 80I, which doubles as a float. Retainer 133I is attached to and extends across a bottom of seal housing 132I to retain float or housing seal 80I. Retainer 133 can also function as a ballast to help control buoyancy and positioning of seal housing 132I within a tank. Both seal 80I and seal housing 132I can be substantially rectangular, cylindrical, or any other suitable shape.

Control valve 36I defines substantially cylindrical air passageway 90I and substantially cylindrical liquid passageway 96I. Air passageway 90I is defined by one or more walls that extend vertically through shoulder 76I and neck 74I of control valve 36G. Inlet 92I of air passageway 90I is the opening located at a bottom end of shoulder 76I. Outlet 94I of air passageway 90I is located at a top end of neck 74I. Liquid passageway 96I is defined mostly by tube 104I. Inlet 98I of liquid passageway 96I extends horizontally into shoulder 76I from liquid inlet port 66I at one side. Once within shoulder 76I, liquid passageway 98I makes an approximately right angled turn to extend vertically out of the bottom end of shoulder 96I and into tank 14. Liquid passageway 98I continues the length of tube 104I to second end 108I, which is attached to nipple 128I. Liquid passageway 98I extends through a center of nipple 128I to outlet 100I located within seal housing 132I. Outlet 100I of liquid passageway 96I is located at shut-off mechanism 68I. Air passageway 90I is several times wider than liquid passageway 96I, and liquid passageway 96I is several times longer than air passageway 90I. As shown in FIG. 12, liquid passageway 96I is located within air passageway 90I when both are extending through shoulder 76I. Control valve 36I is assembled by inserting second end 108I of tube 104I through liquid inlet port 66I and into tank 14I. Second end 108I of tube 104I is attached to nipple 128I on seal housing 132I, and first end 106I of tube 104I extends out of liquid inlet port 66I. Float seal 80I is placed the bottom end of seal housing 132I to function as shut-off mechanism 68I for outlet 100I of liquid passageway 96I.

When control valve 36I is placed in a humidifier tank where liquid levels are relatively high (see FIG. 3), float seal 80I will float on a top surface of the liquid. The flotation of float seal 80I pushes float seal 80I upwardly in seal housing 132I toward outlet 100I of liquid passageway 96I to prevent liquid from exiting control valve 36I. Liquid will flow into inlet 98I and fill passageway 96I/tube 104I, but not exit outlet 100I due to seal formed by float seal 80I. As liquid within the humidifier tank evaporates, float seal 80I will move downwardly along with a surface of depleted liquid. The lowered float seal 80I will drop downwardly within seal housing 132I and no longer press against outlet 100I. Liquid will be free to flow out of outlet 100I and into humidifier tank 14I to replenish the depleted liquid level. Accordingly, shut-off mechanism 68I responds to a level of liquid in humidifier tank by either blocking or allowing incoming liquid flow.

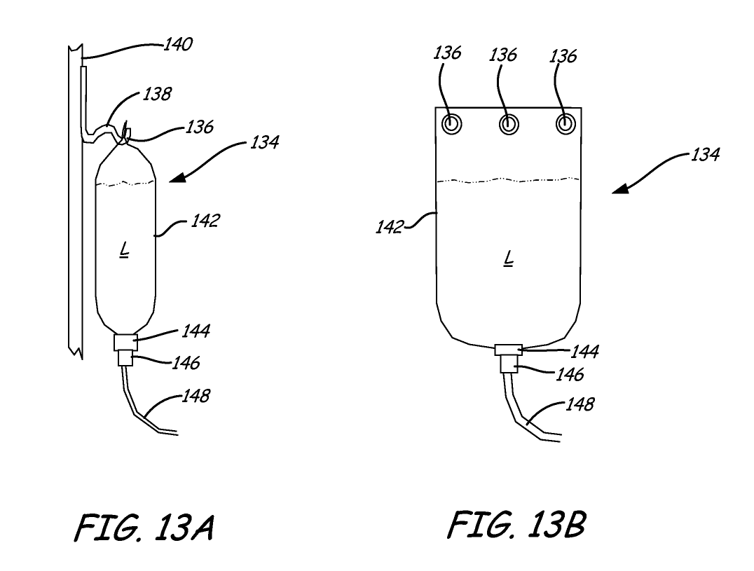

FIG. 13A is a side view, and FIG. 13B is a front view, of flexible auxiliary liquid reservoir 134. Depicted in FIGS. 13A-13B is auxiliary liquid reservoir 134 having grommets 136 for cooperating with hooks 138 on wall 140. Also shown are body 142, reservoir liquid outlet 144, conduit inlet 146, and liquid conduit 148. Auxiliary liquid reservoir 134 is an alternative reservoir for providing liquid to a CPAP humidifier tank.

Auxiliary liquid reservoir 134 is similar to auxiliary reservoir 44 shown in FIG. 1. A top of auxiliary liquid reservoir 134 includes apertures or grommets 136 for receiving hooks 138 extending from wall 140. In alternative embodiments, hooks 138 are attached to a movable stand or any other suitable furniture or surface. Body 142 of auxiliary liquid reservoir 134 is flexible, and can be formed from flexible plastic or the like. Body 142 may resemble an IV bag and contains a volume of liquid L. Liquid L may also include additives such as medicines or aroma therapy. Reservoir liquid outlet 144 is located at a bottom of body 142 and can include a quick connect fixture. Conduit inlet 146 is secured to reservoir liquid outlet 144 and can include a quick connect fixture for easily connecting and forming a fluid connection between auxiliary liquid reservoir 134 and liquid conduit 148.

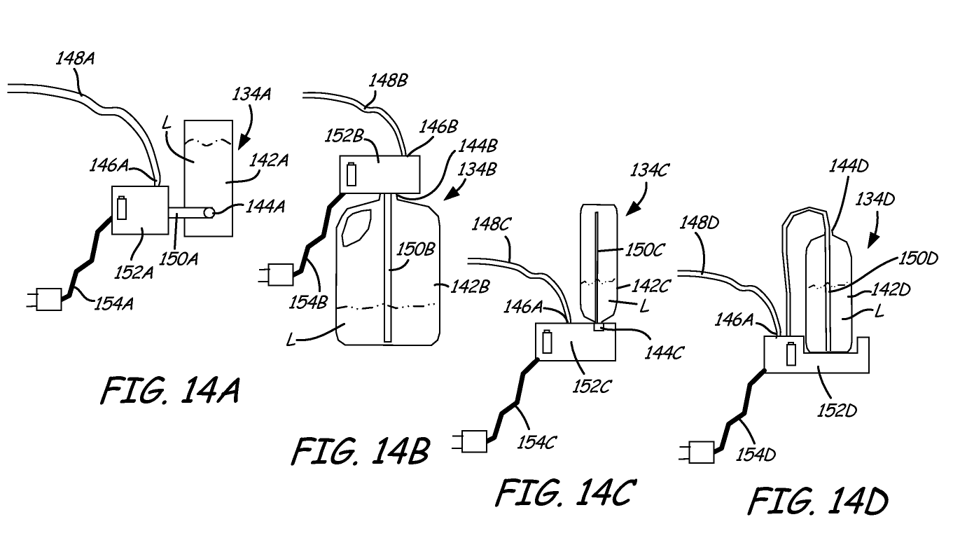

Auxiliary liquid reservoir 134 of FIGS. 13A and 13B functions similarly to auxiliary liquid reservoir 44 of FIG. 1. Liquid L flows from auxiliary reservoir 134 through fluid conduit 148 to a control valve and humidifier tank to replenish liquid in the humidifier tank (e.g. see liquid conduit 38 connecting auxiliary reservoir 44 to control valve 36 and humidifier tank 14). If liquid L contains medication or aroma, then the medication or aroma is also added to the humidifier tank through a control valve. Auxiliary liquid reservoir 134 is hung by grommets 136 a vertical distance about a humidifier to allow gravity to urge liquid L out of auxiliary liquid reservoir 134 through liquid conduit 148 to a control valve and humidifier tank. As described below with reference to FIGS. 14A-14D, auxiliary liquid reservoir can alternatively be attached to a pump.

FIG. 14A is a side view of a first embodiment of auxiliary liquid reservoir 142A and electric pump 152A. Depicted in FIG. 14A are auxiliary liquid reservoir 134A, body 142A, reservoir liquid outlet 144A, conduit inlet 146A, liquid conduit 148A, connecting conduit 150A, electric pump 152A, and electric plug 154A. Auxiliary liquid reservoir 134A is equipped with electric pump 152A for pumping liquid L from auxiliary reservoir 134A to a CPAP humidifier tank.

Auxiliary liquid reservoir 134A is similar to auxiliary reservoir 44 shown in FIG. 1. Body 142A of auxiliary liquid reservoir 134A is pliable but sturdy, can be formed from plastic, and contains liquid L. Reservoir liquid outlet 144A is located at a bottom of body 142A. Connecting conduit 150A is attached to, and fluidly connects, reservoir liquid outlet 144A to a side of electric pump 152A. Auxiliary liquid reservoir 134A is placed side-by-side with electric pump 152A. Conduit inlet 146A of liquid conduit 148A is attached to a top of electric pump 152A. Liquid conduit 148A is intended to fluidly connect electric pump 152A to a control valve secured into a humidifier tank. When connected to a power source such as an outlet, electric plug 154A provides power to electric pump 152A. In alternative embodiments, electric plug 154A is omitted and electric pump 152A includes a battery as a power source.

Auxiliary liquid reservoir 134A of FIG. 14A functions similarly to auxiliary liquid reservoir 44 of FIG. 1. Liquid L flows out of auxiliary reservoir 134A at reservoir liquid outlet 144A, and through connection conduit 150A to electric pump 152A. Electric pump 152A pumps liquid L through conduit inlet 146A and along fluid conduit 148A to a control valve and humidifier tank to replenish liquid in the humidifier tank (e.g. see liquid conduit 38 connecting auxiliary reservoir 44 to control valve 36 and humidifier tank 14 of FIG. 1). Auxiliary liquid reservoir 134A is associated with electric pump 152A to negate the need for gravity in flowing liquid L. In contrast to auxiliary liquid reservoir 134 shown in FIGS. 13A-13B, auxiliary liquid reservoir 134A can be placed anywhere and need not be hung vertically above a CPAP humidifier tank to function as intended. As described below with reference to FIGS. 14B-14D, auxiliary liquid reservoir 134A can be attached to electric pump 152B in a variety of other ways.

FIG. 14B is a side view of a second embodiment of auxiliary liquid reservoir 142B and electric pump 152B. Depicted in FIG. 14B are auxiliary liquid reservoir 134B, body 142B, reservoir liquid outlet 144B, conduit inlet 146B, liquid conduit 148B, connecting conduit 150B, electric pump 152B, and electric plug 154B. Auxiliary liquid reservoir 134B is equipped with electric pump 152B for pumping liquid L from auxiliary reservoir 134B to a CPAP humidifier tank.

Auxiliary liquid reservoir 134B is similar to auxiliary liquid reservoir 44 of FIG. 1 and auxiliary liquid reservoir 134A of FIG. 14A. Body 142B of auxiliary liquid reservoir 134B is pliable but sturdy, can be formed from plastic, and contains liquid L. In the depicted embodiment, auxiliary liquid reservoir 134A is an “off the shelf” gallon jug of water. Reservoir liquid outlet 144B is located at a top of body 142B. Connecting conduit 150B is attached to, and fluidly connects, reservoir liquid outlet 144B to a bottom of electric pump 152B. In the embodiment depicted, connecting conduit 150B extends into body 142B of auxiliary liquid reservoir 134B. Electric pump 152B is located vertically above auxiliary liquid reservoir 134B and a bottom of electric pump 152B can be in contact with a top of auxiliary liquid reservoir 134B. Conduit inlet 146B of liquid conduit 148B is attached to a top of electric pump 152B. Liquid conduit 148B is intended to fluidly connect electric pump 152B to a control valve to a humidifier tank. When connected to a power source such as an outlet, electric plug 154B provides power to electric pump 152B. In alternative embodiments, electric plug 154B is omitted and electric pump 152B includes a battery as a power source.

Auxiliary liquid reservoir 134B of FIG. 14B functions similarly to auxiliary liquid reservoir 44 of FIG. 1 and auxiliary liquid reservoir 134A of FIG. 14A. Liquid L is pulled out of auxiliary reservoir 134B at reservoir liquid outlet 144B by connection conduit 150A and into electric pump 152A. Electric pump 152B pumps liquid L through conduit inlet 146B and along fluid conduit 148B to a control valve and humidifier tank to replenish liquid in the humidifier tank (e.g. see liquid conduit 38 connecting auxiliary reservoir 44 to control valve 36 and humidifier tank 14 in FIG. 1). Auxiliary liquid reservoir 134B is associated with electric pump 152B to negate the need for gravity in flowing liquid L to a CPAP humidifier tank. In contrast to auxiliary liquid reservoir 134 shown in FIGS. 13A-13B, auxiliary liquid reservoir 134B can be placed anywhere and need not be hung vertically above a CPAP humidifier tank to function as intended. As described below with reference to FIGS. 14C-14D, auxiliary liquid reservoir 134B can be attached to electric pump 152B in a variety of other ways.

FIG. 14C is a side view of a third embodiment of auxiliary liquid reservoir 142C and electric pump 152C. Depicted in FIG. 14C are auxiliary liquid reservoir 134C, body 142C, reservoir liquid outlet 144C, conduit inlet 146C, liquid conduit 148C, connecting conduit 150C, electric pump 152C, and electric plug 154C. Auxiliary liquid reservoir 134C is equipped with electric pump 152C for pumping liquid L from auxiliary reservoir 134C to a CPAP humidifier tank.

Auxiliary liquid reservoir 134C is similar to auxiliary liquid reservoir 44 of FIG. 1, auxiliary liquid reservoir 134A of FIG. 14A, and auxiliary liquid reservoir 134B of FIG. 14B. Body 142C of auxiliary liquid reservoir 134C is pliable but sturdy, can be formed from plastic, and contains liquid L. In the depicted embodiment, auxiliary liquid reservoir 134B is an “off the shelf” bottle of water. Reservoir liquid outlet 144C is located at a bottom of body 142C. Connecting conduit 150C is attached to, and fluidly connects, reservoir liquid outlet 144C to a top of electric pump 152C. In the embodiment depicted, connecting conduit 150C extends upwardly into body 142C of auxiliary liquid reservoir 134C. Electric pump 152C is located vertically below auxiliary liquid reservoir 134C and a top of electric pump 152C can be in contact with a bottom of auxiliary liquid reservoir 134C. Conduit inlet 146C of liquid conduit 148C is attached to a top of electric pump 152C next to auxiliary liquid reservoir 164C. Liquid conduit 148C is intended to fluidly connect electric pump 152C to a control valve attached to a humidifier tank. When connected to a power source such as an outlet, electric plug 154C provides power to electric pump 152C. In alternative embodiments, electric plug 154C is omitted and electric pump 152C includes a battery as a power source.

Auxiliary liquid reservoir 134C of FIG. 14C functions similarly to auxiliary liquid reservoir 44 of FIG. 1, auxiliary liquid reservoir 134A of FIG. 14A, and auxiliary liquid reservoir 134B of FIG. 14B. Liquid L flows out of auxiliary reservoir 134C at reservoir liquid outlet 144C by connection conduit 150C and into electric pump 152C. Electric pump 152C pumps liquid L through conduit inlet 146C and along fluid conduit 148C to a control valve and humidifier tank to replenish liquid in the humidifier tank (e.g. see liquid conduit 38 connecting auxiliary reservoir 44 to control valve 36 and humidifier tank 14 in FIG. 1). Auxiliary liquid reservoir 134C is associated with electric pump 152C to negate the need for gravity in flowing liquid L to a CPAP humidifier tank. In contrast to auxiliary liquid reservoir 134 shown in FIGS. 13A-13B, auxiliary liquid reservoir 134C can be placed anywhere and need not be hung vertically above a CPAP humidifier tank to function as intended. As described below with reference to FIG. 14D, auxiliary liquid reservoir 134C can be attached to electric pump 152C in other ways.

FIG. 14D is a side view of a fourth embodiment of auxiliary liquid reservoir 142D and electric pump 152D. Depicted in FIG. 14D are auxiliary liquid reservoir 134D, body 142D, reservoir liquid outlet 144D, conduit inlet 146D, liquid conduit 148D, connecting conduit 150D, electric pump 152D, and electric plug 154D. Auxiliary liquid reservoir 134D is equipped with electric pump 152D for pumping liquid L from auxiliary reservoir 134D to a CPAP humidifier tank.

Auxiliary liquid reservoir 134D is similar to auxiliary liquid reservoir 44 of FIG. 1, auxiliary liquid reservoir 134A of FIG. 14A, auxiliary liquid reservoir 134B of FIG. 14B, and auxiliary liquid reservoir 134C of FIG. 14C. Body 142D of auxiliary liquid reservoir 134D is pliable but sturdy, can be formed from plastic, and contains liquid L. In the depicted embodiment, auxiliary liquid reservoir 134D is an “off the shelf” bottle of water. Reservoir liquid outlet 144D is located at a top of body 142D. Connecting conduit 150D is attached to, and fluidly connects, reservoir liquid outlet 144D to a top of electric pump 152D. In the embodiment depicted, connecting conduit 150D extends upwardly from electric pump and then downwardly into body 142D of auxiliary liquid reservoir 134D. Electric pump 152D includes a space for supporting auxiliary liquid reservoir 134D, such that a top electric pump 152D can be in contact with a bottom of auxiliary liquid reservoir 134D. Conduit inlet 146D of liquid conduit 148D is attached to a top of electric pump 152D next to auxiliary liquid reservoir 134D. Liquid conduit 148D is intended to fluidly connect electric pump 152D to a control valve attached to a humidifier tank. When connected to a power source such as an outlet, electric plug 154D provides power to electric pump 152D. In alternative embodiments, electric plug 154D is omitted and electric pump 152D includes a battery as a power source.

Auxiliary liquid reservoir 134D of FIG. 14D functions similarly to auxiliary liquid reservoir 44 of FIG. 1, auxiliary liquid reservoir 134A of FIG. 14A, auxiliary liquid reservoir 134B of FIG. 14B, and auxiliary liquid reservoir 134C of FIG. 14C. Liquid L is pulled out of auxiliary reservoir 134D at reservoir liquid outlet 144D by connection conduit 150D and into electric pump 152D. Electric pump 152D pumps liquid L through conduit inlet 146D and along fluid conduit 148D to a control valve and humidifier tank to replenish liquid in the humidifier tank (e.g. see liquid conduit 38 connecting auxiliary reservoir 44 to control valve 36 and humidifier tank 14 in FIG. 1). Auxiliary liquid reservoir 134D is associated with electric pump 152D to negate the need for gravity in flowing liquid L to a CPAP humidifier tank. In contrast to auxiliary liquid reservoir 134 shown in FIGS. 13A-13B, auxiliary liquid reservoir 134D can be placed anywhere and need not be hung vertically above a CPAP humidifier tank to function as intended.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A respiratory humidifier comprising:
a tank storing liquid;
an air inlet opening located on the tank and fluidly connecting the tank to an air pump;
an air outlet opening located on the tank and fluidly connecting the tank to an air mask; and
a control valve attached to the air outlet opening and defining both an air outlet passageway and a liquid inlet passageway, the control valve including a shut-off mechanism for blocking the liquid inlet passageway when the liquid in the tank reaches a level.

2. The respiratory humidifier of claim 1, wherein the shut-off mechanism includes a float that blocks the liquid inlet passageway when in sufficient contact with a top surface of liquid in the tank.

3. The respiratory humidifier of claim 1, wherein the control valve has an interference fit with the air outlet opening of the tank.

4. The respiratory humidifier of claim 1, wherein a portion of the air outlet passageway and a portion of the liquid inlet passageway extend in parallel within the control valve.

5. The respiratory humidifier of claim 4, wherein a portion of the air outlet passageway surrounds a portion of the liquid inlet passageway.

6. The respiratory humidifier of claim 1, wherein the control valve further comprises:
a top portion that defines a first end of the air outlet passageway;
a liquid inlet port extending into the top portion, the liquid inlet port defining a first end of the liquid inlet passageway;
a fitting portion attached to the top portion, the fitting portion securing the control valve to the air outlet opening; and
a bottom portion attached to the fitting portion, the bottom portion defining a second end of the air outlet passageway and a second end of the liquid inlet passageway, wherein the shut-off mechanism is attached to the second end of the liquid inlet passageway.

7. The respiratory humidifier of claim 6, wherein the liquid inlet port extends into a side of the top portion.

8. The respiratory humidifier of claim 6, wherein the bottom portion extends through the air outlet opening of the tank.

9. The respiratory humidifier of claim 6, wherein the bottom portion includes vents for receiving air into the second end of the air outlet passageway.

10. The respiratory humidifier of claim 6, wherein the fitting portion includes a lip that abuts the air outlet opening.

11. The respiratory humidifier of claim 6, wherein the fitting portion extends over the air outlet opening.

12. A CPAP humidifier system comprising:
an air pump;
a humidifier tank having an air inlet opening and an air outlet opening, the air inlet opening fluidly connected to the air pump and the air outlet opening fluidly connected to an air mask;
a control valve attached to the air outlet opening and defining both an air outlet passageway and a liquid inlet passageway; and
an auxiliary reservoir fluidly connected to the liquid inlet passageway for providing liquid to the humidifier tank.

13. The CPAP humidifier system of claim 12, further comprising:
an electric pump attached to the auxiliary reservoir.

14. The CPAP humidifier system of claim 12, wherein the auxiliary reservoir is a flexible water container positioned above the humidifier tank.

15. The CPAP humidifier system of claim 14, wherein the flexible water container includes an additive.

16. The CPAP humidifier system of claim 12, wherein the control valve includes a float responsive to liquid levels in the humidifier tank.

17. A method of providing humidified air to a user, the method comprising:
pumping air;
flowing pumped air to a humidifier tank to form humidified air;
flowing humidified air from the humidifier tank through a control valve to a mask; and
flowing liquid from an auxiliary tank through the control valve to the humidifier tank.

18. The method of claim 17, wherein humidified air flows through the control valve and liquid flows through the control valve simultaneously.

19. The method of claim 17, further comprising:
regulating the flow of liquid through the control valve based on liquid level in the humidifier tank.

20. The method of claim 17, wherein gravity causes the flow of liquid from the auxiliary tank.
